(19) United States
(12) Patent Application Publication
(10) Pub. No.: US 2008/0039531 A1
(43) Pub. Date: Feb. 14, 2008

(54) Compositions, and kits comprising targeted aldehyde or acetal protease inhibitor compounds for treating muscle disorders

(76) Inventors: Alfred Stracher, Roslyn, NY (US); Leo Kesner, West Orange, NJ (US); Ted Carver, Charlotte, NC (US); Norman W. Barton, Phoenix, MD (US)

Correspondence Address: NEEDLE & ROSENBERG, P.C., SUITE 1000, 999 PEACHTREE STREET, ATLANTA, GA, 30309-3915, US

(21) Appl. No.: 11/321,782

(22) Filed: Dec. 28, 2005

Related U.S. Application Data
(63) Continuation-in-part of application No. PCT/US05/20903, filed on Jun. 13, 2005.

(51) Int. Cl. A61K 31/11; A61P 21/00

(52) U.S. Cl. 514/693

(57) ABSTRACT

Described herein are compounds that comprise a carrier residue that is or is an analog of carnitine, an optional linker group, and a residue of a protease inhibitor comprising an aldehyde group or acetal derivatives of the aldehyde group, and pharmaceutical compositions and kits thereof. The compounds, compositions, and kits are useful for treating muscle disorders in animals and humans that result from the undesired activity of biological proteases. The compounds of the invention are particularly effective as calpain inhibitors, and can be used to treat the resulting muscular disorders, exemplified by Duchenne or Becker muscular dystrophy.

### CROSS REFERENCE TO RELATED APPLICATIONS

This application claims the priority of and is a continuation-in-part of PCT International Patent Application Serial No. PCT/US05/20903, filed Jun. 13, 2005, and also claims the priority of U.S. provisional application Ser. Nos. 60/578,914 and 60/633,274, filed on Jun. 12, 2004 and Dec. 3, 2004, respectively. These applications are hereby all incorporated by this reference in their entireties for all of their teachings, for all purposes.

### BACKGROUND

The treatment of muscle disorders is a growing field of medical technology. Muscle disorders, including many known degenerative muscle disorders, can have a variety of causes, including genetic defects, metabolic disorders, trauma, ischemia, sepsis, or cancer, and can be very debilitating and can substantially reduce the quality of life of the patient. See for example Wagner K R “Genetic Diseases of Muscle” Neurol Clin. 2002 August; 20(3):645-78, discussing various forms of muscular dystrophy, and Hasselgren P O, Fischer J E “Muscle Cachexia: Current Concepts of Intracellular Mechanisms and Molecular Regulation,” Ann Surg. 2001 January; 233(1):9-17, discussing the role of myofibrillar protein catabolism and/or degradation in muscle cachexia induced by severe injury, sepsis, and cancer.

Various naturally occurring protease enzymes are believed to be part of the basic machinery of cellular catabolism and part of the mechanism of cell death pathways. See Nakanishi, H. “Involvement of Neuronal and Microglial Proteinases in Neuronal Death,” Neurochem 1999; 2: 217-32. The relevant proteases include the classes of serine, threonine, and cysteine proteases, wherein it is believed that an oxygen or sulfur atom from a serine, threonine, or cysteine amino acid at the active site serves as a nucleophile to cleave the amide bonds of the protein.

Calcium-activated neutral proteases (“Calpains”) are a family of cysteine proteases whose proteolytic activity is accelerated when abnormally high amounts of Ca⁺² enter the cell by virtue of increased membrane permeability, as a result of various defects including a traumatic or ischemic event and/or a genetic defect. Calpains are structurally related heterodimeric neutral and non-lysosomal Ca²⁺ activated cysteine proteases that are found in all tissue and cell types. Calpains are active in promoting programmed cell death, or apoptosis, and have been implicated in the initiation of both necrotic and apoptotic cell death. Calpains are also believed to be involved in important signaling pathways, modulation of enzyme activity, processing of hormones, protein turnover, and cytoskeletal rearrangements. See “The Calpain System” by Goll et al., Physiol Rev. 2003 July; 83(3):731-801), and Ray et al., (“Calpain and Its Involvement in the Pathophysiology of CNS Injuries and Diseases. Therapeutic Potential of Calpain Inhibitors for Prevention of Neurodegeneration,” Current Drug Targets-CNS and Neurological Disorders, 2, 173-189, 2003), Perrin B J, Huttenlocher A. “Calpain,” Int J Biochem Cell Biol. 2002 July; 34(7):722-5; and Zatz M. and Starling A “Calpains and Disease,” New England Journal of Medicine 2005, June; 352(23): 2413-2423.

Over the past ten years, it has emerged that calpain enzymatic activity plays a key role in a very large number of cellular degenerative conditions. Calpains are believed to be involved in many cellular death processes initiated by various traumas, ischemia, and immunological and inflammation-induced diseases, as well as a variety of central nervous system diseases. In many of the relevant diseases, various cellular and/or membrane defects and/or injuries affect the cellular homeostasis of calcium ions, leading to increased concentrations of intracellular calcium ions and the activation of the calpains. See Vanderklish P W, Bahr B A “The Pathogenic Activation of Calpain: a Marker and Mediator of Cellular Toxicity and Disease States” Int. J. Exp. Pathol., 2000 October; 81(5):323-39; Wang K K, and Yuen P W “Calpain Inhibition: An Overview of Its Therapeutic Potential,” Trends Pharmacol. Sci., 1994 November: 15(11):412-9. Tissues weakened by ischemia/reperfusion injury, such as occurs following stroke or myocardial infarct, admit Ca⁺² ions. Calpains become activated against a number of cytosolic and membrane proteins, cytokines, transcriptional factors, kinases, phosphatases, and lens proteins, when increased levels of intracellular free Ca²⁺ ions are present (micromolar levels for “μcalpains” and millimolar concentrations for “m-calpains”).

For example, it is believed that in muscle cachexia, the early release of myofilaments from muscular myofibrils is initiated by calpains from the cysteine protease family, then the myofibrils generated are ubiquitinated and further degraded by proteosomes. See Hasselgren et al, page 13-14, and the description of proteosomes by Lee D. H., and Goldberg A. L. “Proteasome Inhibitors: Valuable New Tools for Cell Biologist,” Cell Biology, 8, 397-399, 1998. In many other degenerative muscle diseases, it is believed that calpains are the primary proteases that initiate the degradation of muscle.

Calpains are also believed to be involved in Duchenne and Becker muscular dystrophies. As well known to those of ordinary skill in the art, and as described by Wagner, Duchenne muscular dystrophy (“DMD”) is an inherited and lethal disease of boys that is estimated to effect 1 in 3500 live male births. The male child typically comes to medical treatment at the age of 3 to 5 years, because of frequent falls, slow running, and waddling gait. The disease and resulting muscular weakness, progresses relentlessly with loss of ambulation between 7 and 12 years of age. Prednisone, the primary currently known pharmacological therapy for DMD can modestly prolong ambulation, but death occurs in the late teens to early twenties due to respiratory or cardiac complications. Becker muscular dystrophy, (“BMD”) is an allelic variant of DMD, has a more benign and variable presentation, with later onset and slower progression.

The dystrophin gene and corresponding protein responsible for DMD and BMD is believed to be involved in maintaining cell membrane integrity and/or regulating calcium “leakage” through the cell membranes. See Stracher, “Calpain Inhibitors as Therapeutic Agents in Nerve and Muscle Degeneration,” Ann. New York Acad. Sci., 1999, 884, 52-59. When dystrophin is mutated or absent, the membrane often becomes more permeable to calcium ions. The resulting activation of calpains is believed to initiate the myofibrillar protein degradation observed in muscular dystrophy, as is also observed in the well known mdx rat model of muscular dystrophy.

Small molecule calpain inhibitors are known and have been described in numerous scientific publications and patent literature. See Wang K K, and Yuen P W, “Development and Therapeutic Potential of Calpain Inhibitors,” Advances in Pharmacology, 1997, 37, 117-152; Wang, K. K. et al “An Alpha-mercaptoacrylic acid a Selective Non-peptide Cell Permeable Calpain Inhibitor and is Neuroprotective” in Proc. Natl. Acad. Sci. USA, volume 93, pages 6687-6692 (1996); Hernadez et al., “Recent Advances in the Synthesis, Design and Selection of Cysteine Protease Inhibitors,” Curr. Opin. Chem. Biol., 2002 August; 6(4):459-65; and DePetrillo, P. B., “Calpain Inhibitors—A Review of the Recent Patent Literature” Idrugs 2002, 5(6), 568-576. See also U.S. Pat. No. 5,081,204 (Higuchi), U.S. Pat. No. 5,486,623 (Zimmerman), U.S. Pat. No. 5,498,616 (Mallamo), U.S. Pat. No. 5,506,243 (Ando), and U.S. Pat. No. 5,514,694 (Powers). additional examples of calpain inhibitors in the patent literature include WIPO Publication Nos. WO 92/11850 (Cortex Pharmaceutical), WO 94/00095 (Cortex) and WO 95/00535 (Alkermes Inc.).

The references above describe a variety of different chemical classes and/or chemical entities for the inhibition of calpains, including peptide keto compounds, peptide aldehydes and alpha-ketoamides, N-substituted peptidyl compounds, peptidyl ketone heterocyclic ethers, heterocyclic-N-heteroatom methyl ketones, sulfonamide pyrrolidines, and peptidyl ketoamides. The peptide aldehyde class of calpain inhibitors, which substitute the terminal carboxyl group of a peptide with an aldehyde group, are believed to reversibly inhibit calpains by reacting with the active site thiol of the enzyme to reversibly form a thiohemiacetal. Nevertheless, peptide aldehydes and their close analogues often have relatively poor cell permeability due to their high polarity, are not very stable to peptidase degradation, and are not very selective for calpains, typically also inhibiting other serine and cysteine proteases in the body, such as papain, trypsin, and cathepsin B, as well as the threonine proteases within proteosomes. See Lee and Goldberg, and DePetrillo (2002). Attempts to improve cell permeability of peptide aldehydes and analogs thereof by capping or otherwise modifying the N-terminal group of the peptide aldehydes led to improvements in activity, but “The compounds however, still lack specificity and are readily oxidized under physiological conditions because they are aldehydes.” See Wang and Yuen, 1997, and Donkor, I. O., Current Medicinal Chemistry, 2000, 7:1171-1188.

Leupeptin, an acetylated tripeptide aldehyde comprising two normal leucyl amino acids, and one “argininal” residue that is an aldehyde analog of arginine. Leupeptin can be isolated from certain streptomyces strains, and is a potent inhibitor of the calpain class of enzymes in animal models based on the inhibitory effect it exerts on myofiber degradation. See Badalamente M, Stracher A. “Delay of Muscle Degeneration and Necrosis in Mdx Mice by Calpain Inhibition.” Muscle and Nerve 2000; 23:106-11; Badalamente M, Hurst, L., and Stracher A, “Neuromuscular Recovery After Peripheral Nerve Repair Effects of an Orally Administered Peptide in a Primate Model,” J. Reconstructive Microsurgery, 1995, 11(6), 429-437, and Stracher, 1999. Leupeptin's structure is shown below:

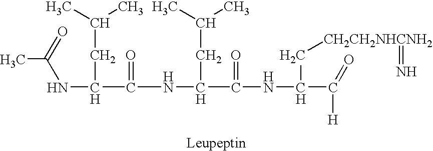

Among the well known limitations of leupeptin and other related peptide aldehydes and their close analogs are poor cellular permeabilities due to high polarity, poor chemical and stereochemical stability, and undesirably rapid in-vivo oxidation of the aldehyde groups to the inactive carboxylic acids.

Leupeptin and almost all other know protease and/or calpain inhibitors, whether administered either orally or parenterally, distribute themselves indiscriminately in many non-diseased tissues in the body, when ideally only muscle tissue should be targeted. Moreover, because leupeptin and other known small molecule protease inhibitors are not very selective, they typically inhibit a variety of proteases, including serine proteases and other cysteine proteases. As a result, relatively high doses may be required for effective treatment, which is expensive and can aggravate potential dose-limiting side effects in other parts of the body.

A family of patents exemplified by U.S. Pat. Nos. 4,742,081, 4,866,040, 5,008,288, and 5,876,747 disclosed an attempt to bond various protease inhibitors, through optional linker groups, to “carrier” residues that could potentially selectively transport and/or concentrate the protease inhibitor in desired sites in the body, such as skeletal muscle or the heart. The carrier residues disclosed included carnitine, aminocarnitine, and an analog of taurine thereof, cysteic acid. Carnitine and its derivative carboxylate esters are well known to be involved in active biological transport of fatty acids across cellular membranes in muscle tissues. One of the many protease inhibitor residues disclosed in the referenced patents was the peptide aldehyde residue leucyl argininal. A compound comprising aminocarnitine and leucyl argininal, linked by a glutaric acid residue was disclosed, and was synthesized via use of a dibutyl acetal synthetic intermediate, but neither the final peptide aldehyde compound, nor any other of the disclosed and/or suggested protease inhibitor compounds were suggested to be effective in the treatment of any specific disease, or actually demonstrated to be effective for the treatment of any particular disease. Moreover, there was no disclosure or suggestion that the acetal synthetic intermediates would have any pharmaceutical activity at all.

Overall, while the disclosures described above illustrate that many attempts have been made to develop clinically effective calpain inhibitors, for use in treatment of a variety of diseases, progress has been limited. As noted by Donkor, “A variety of peptidyl calpain inhibitors derived from mammalian, plant, and synthetic sources have appeared in the literature. The Majority of these inhibitors . . . [have] . . . limited selectivity for calpain over other cysteine proteases . . . . Though potency enhancement has been realized to some degree, selectivity via this approach still remains a formidable challenge.” Furthermore, as noted by DePetrillo, “However, issues of stability and delivery point to non-peptide inhibitors as promising agents, since they might reach target sites more effectively after oral administration, while peptidyl inhibitors and peptidomimetic inhibitors might be expected to require parenteral delivery since they could be more prone to tissue aminopeptidase-induced degradation.” Accordingly, the teachings of the literature have tended to lead those of ordinary skill in the art away from peptide aldehyde analogs such as the compounds of the invention disclosed herein.

In the particular cases of Duchenne and Becker muscular dystrophies, many failed attempts have been been made to develop clinical acceptable treatments. As noted above, prednisone, the primary currently known pharmacological therapy, only modestly delays the progression of symptoms, and does not prevent eventual death of the patient. Recent attempts to develop a treatment for DMD by administration of aminoglycoside anti-biotics, in hopes of increasing the expression of dystrophin failed. See Dunant et al., “Gentamycin Fails to Increase Dystrophin Expression in Dystrophin-Deficient Muscle,” Mucle and Nerve, 2003, 27, 624-627.

Thus, there remains a long felt and unmet need in the art for compounds and methods to target more directly muscle tissue and protease enzymes such as calpains with greater efficacy, at lower dosages and higher safety margin, for application to the treatment of degenerative muscular diseases such as Duchenne muscular dystrophy.

### SUMMARY

Described herein are compounds, pharmaceutical compositions, and kits for treating muscle disorders and methods of use thereof. It has been discovered that certain protease inhibitors, including peptide aldehyde compounds, can be directly or indirectly bonded (through optional “linker” groups), to “carrier” molecules that include analogs of carnitine, and that the resulting analogs of compounds are effective calpain inhibitors that can be selectively transported into or concentrated in muscle tissues, so that the clinical effects of any lack of selectivity for the inhibition of calpains, and any associated side effects are minimized. Moreover, it has been discovered that the use of “acetal prodrugs” of the aldehyde compounds of the invention can be readily and advantageously converted, immediately prior to or during administration, to the active aldehyde form of the compound that is an effective protease inhibitor. Even more surprisingly, it has been discovered that the “acetal prodrug” form of the parent active aldehydes can be directly administered to patients, efficiently absorbed and selectively concentrated in muscle tissues, and can be converted to the active aldehyde form of the compound in-vivo, so as to inhibit calpains in the muscular tissues involved in the muscle disorders, including Duchenne and Becker muscular dystrophies.

The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below. Like numbers represent the same elements throughout the figures.

FIG. 1 depicts a reaction scheme for synthesizing aminocarnityl-succinyl-leucyl-argininal.

FIG. 2 shows the absorption of leupeptin (18 mg/kg) into plasma after intramuscular injection in hind-limbs of rats, which shows a peak of 4.5 μg/ml at 1 hr and a return to 0 by 24 hr, as described in Example 1.

FIG. 3 shows the quantification of myofiber diameters, which shows statistically significant increased diameters in the right opponens and abductor muscles of treated animals, at both 12 and 24 weeks, after delayed nerve repair. Normal myofiber diameter, obtained as a mean in the left, unoperated hand of control animals, is shown by open arrows on the left of graph.

FIG. 4 shows the mean myofiber diameters in the left and right opponens pollicis muscles 3 months after right median nerve repair. On the right, all leupeptin treatment groups show a significant increase compared to the right, control group (*p<0.01).

FIG. 5 shows mean myofiber diameters in the left and right abductor pollicis muscles 6 months after right median nerve repair. On the left, the 6-mg, 12-mg, and 18-mg leupeptin treatment groups show a small but significant increase, compared to the left, control group (*p<0.01). On the right, the 6-mg, 12-mg, and 18-mg leupeptin-treatment groups show a significant increase, compared to the right, control group (*p<0.01).

FIG. 6 shows the appearance of spectrin breakdown products in RD cells that have been incubated with maitotoxin in the presence of calcium.

FIG. 7 shows results of the comparative calpain inhibition activity of CLA aldehyde enantiomers prepared from the corresponding L and D argininal enantiomers as described in Example 12.

FIG. 8 shows a generic method of preparing the compounds of the invention by condensing peptide aldehyde acetals with dialdehydes, subsequent condensation with aminocarnitine, and reduction of the resulting di-imine intermediates.

FIG. 9 shows a generic method of preparing the compounds of the invention by condensing peptide aldehyde acetals with succinic anhydride, and subsequent condensation of the resulting intermediate with carnitine or aminocarnitine.

### DETAILED DESCRIPTION

Before the present compounds, compositions, articles, devices, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

### DEFINITIONS

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or can not be substituted and that the description includes both unsubstituted lower alkyl and lower alkyl where there is substitution.

Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

References in the specification and concluding claims to parts by weight, of a particular element or component in a composition or article, denote the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

Variables such as R¹-R⁶, R¹⁰, m, n, o, X, Y, and Z used throughout the application are the same variables as previously defined unless stated to the contrary.

The term “alkyl group” as used herein is a branched or unbranched saturated hydrocarbon group of 1 to 25 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms or one to four carbon atoms.

The term “polyalkylene group” as used herein is a group having two or more CH₂ groups linked to one another. The polyalkylene group can be represented by the formula —(CH₂)ₙ—, where n is an integer of from 2 to 25.

The term “polyether group” as used herein is a group having the formula —[(CHR)ₙO]ₘ—, where R is hydrogen or a lower alkyl group, n is an integer of from 1 to 20, and m is an integer of from 1 to 100. Examples of polyether groups include, polyethylene oxide, polypropylene oxide, and polybutylene oxide.

The term “polythioether group” as used herein is a group having the formula —[(CHR)ₙS]ₘ—, where R is hydrogen or a lower alkyl group, n is an integer of from 1 to 20, and m is an integer of from 1 to 100.

The term “polyimino group” as used herein is a group having the formula —[(CHR)ₙNR]ₘ—, where each R is, independently, hydrogen or a lower alkyl group, n is an integer of from 1 to 20, and m is an integer of from 1 to 100.

The term “polyester group” as used herein is a group that is produced by the reaction between a compound having at least two carboxylic acid groups with a compound having at least two hydroxyl groups.

The term “polyamide group” as used herein is a group that is produced by the reaction between a compound having at least two carboxylic acid groups with a compound having at least two unsubstituted or monosubstituted amino groups.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

By “subject” is meant an individual. Preferably, the subject is a mammal such as a primate, and, more preferably, a human. The term “subject” can include domesticated animals, such as cats, dogs, etc., livestock (e.g., cattle, horses, pigs, sheep, goats, etc.), and laboratory animals (e.g., mouse, rabbit, rat, guinea pig, etc.).

By “contacting” is meant an instance of exposure by close physical contact of at least one substance to another substance. For example, contacting can include contacting a substance, such as a pharmacologic agent, with a cell. A cell can be contacted with a test compound, for example, a context dependent inhibitor, or putative context dependent inhibitor by adding the agent to the culture medium (by continuous infusion, by bolus delivery, or by changing the medium to a medium that contains the agent) or by adding the agent to the extracellular fluid in vivo (by local delivery, systemic delivery, intravenous injection, bolus delivery, or continuous infusion). The duration of contact with a cell or group of cells is determined by the time the test compound is present at physiologically effective levels or at presumed physiologically effective levels in the medium or extracellular fluid bathing the cell.

“Induce” means initiating a desired response or result that was not present prior to the induction step. The term “potentiate” means sustaining a desired response at the same level prior to the potentiating step or increasing the desired response over a period of time.

“Treatment” or “treating” means to administer a composition to a subject or a system with an undesired condition or at risk for the condition. The condition can include a disease or a predisposition to a disease. The effect of the administration of the composition to the subject can have the effect of but is not limited to reducing or preventing the symptoms of the condition, a reduction in the severity of the condition, or the complete ablation of the condition.

The term “protecting group” as used herein is a group that can be chemically bound to an atom or molecule, so as to allow a subsequent reaction to proceed as desired, without interference with or destruction of desirable molecular features, and is subsequently removed (either chemically, in-vitro, or in-vivo) from the atom or molecule by predictable methods to regenerate a desired modified molecule that does not comprise the protecting group. Examples of many of the possible protective groups can be found in Protective Groups in Organic Synthesis by T. W. Green, John Wiley and Sons, 1981, which is incorporated herein by reference in its entirety.

Any of the compounds described herein can exist as a prodrug. The term “prodrug” is defined herein as an inactive form of a parent drug that has been created to overcome one or more barriers to their effective use. For example, a prodrug can be a compound that has a protecting group or other organic functional group that is cleaved upon administration to a subject to release the active form of the drug. In certain aspects, if the active compound is unstable, it can be prepared as its salt form in order to increase stability in dry form (e.g., powder). Methods for converting prodrugs of the compounds described herein will be discussed in greater detail below.

In examples well known to those of ordinary skill in the art, pharmaceutically active compounds having an alcohol (—OH) group or a carboxylic acid group (a —CO₂H group, such as the —CO₂H group of the carrier molecules of the present invention) can be readily chemically modified to form the corresponding carboxylic acid ester prodrugs (having a-CO₂R group). Such prodrugs, known as ester prodrugs, which may or may not be pharmaceutically active themselves, can often be designed to exhibit improved general absorption and/or biological membrane permeability as compared to the parent pharmaceutically active compound, but the prodrug group is readily cleaved in-vivo after administration to liberate the parent pharmaceutically active compound, and thus improves the general bioavailability of the parent pharmaceutically active compound.

Many of the pharmaceutically active protease inhibitor compounds of the present invention can comprise an aldehyde group, and prior to administration to the subject such aldehyde compound can be reacted with alcohols to form an “acetal prodrug” of the aldehyde by methods well known to those of ordinary skill in the art. Such acetal prodrug compounds can exhibit improved chemical and/or stereochemical stability as compared to the aldehyde active parent compounds, both during synthesis and storage, and prior to, during, or after administration to a subject. The acetal groups can be readily hydrolyzed in-vitro by many known methods, especially by treatment with aqueous acids, and/or mild heating, to liberate the parent aldehyde just prior to administration of the aldehyde to the subject:

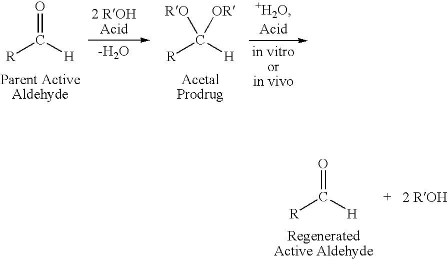

The absorption and/or bio-availability of acetal prodrug forms of the aldehyde drugs described herein can be better than the parent aldehydes. Moreover, it has been unexpectedly discovered that such aldehyde prodrugs can be administered to a subject and be hydrolyzed in-vivo to release the biologically active parent aldehyde compound.

Also, one or more compounds disclosed herein can include zwitterionic salts formed by reaction of a nitrogen contained internally within the compound, such as an amine, aniline, pyridyl, arginine, and like residues with an acidic hydrogen within the compound, such as the sulfonic acid groups. Alternatively, a basic nitrogen contained internally within the compound, such as the arginine or amine groups or residues can be reacted with a pharmaceutically acceptable external acid, such as HCl, sulfuric acid, a carboxylic acid or the like.

By “pharmaceutically acceptable” is meant a material that is not biologically or otherwise undesirable, i.e., the material can be administered to an individual along with the relevant active compound without causing clinically unacceptable biological effects or interacting in a deleterious manner with any of the other components of the pharmaceutical composition in which it is contained.

By the term “effective amount” of a compound as provided herein is meant a sufficient amount of the compound or composition to provide the desired regulation of a desired function or outcome, such as protein activation or inhibition, or a disease condition or one of the physical or clinical manifestations thereof. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The term “residue” as used herein refers to the moiety that is the resulting product of the chemical species in a particular reaction scheme or subsequent formulation or chemical product, regardless of whether the moiety is actually obtained from the chemical species. For example, a linker L that contains at least one —OH group can be represented by the formula L-OH, where L is the remainder (i.e., residue) of the linker.

The term “organic residue” defines a carbon containing residue, i.e. a residue comprising at least one carbon atom, and includes but is not limited to the carbon-containing groups, residues, or radicals defined hereinabove. Organic residues can contain various heteroatoms, or be bonded to another molecule through a heteroatom, including oxygen, nitrogen, sulfur, phosphorus, or the like. Examples of organic residues include but are not limited to alkyl or substituted alkyls, alkoxy or substituted alkoxy, mono or di-substituted amino, amide groups, etc. Organic resides can preferably comprise 1 to 18 carbon atoms, 1 to 15 carbon atoms, 1 to 12 carbon atoms, 1 to 8 carbon atoms, or 1 to 4 carbon atoms.

A very close synonym of the term “residue” is the term “radical,” which as used in the specification and concluding claims, refers to a fragment, group, or substructure of a molecule described herein, regardless of how the molecule is prepared. In some embodiments the radical (for example an alkyl) can be further modified (i.e., substituted alkyl) by having bonded thereto one or more “substituent radicals.”

The term “compound” as used herein also includes corresponding prodrugs of the compounds of the invention, including actetal prodrugs, and/or one or more pharmaceutically-acceptable salts or esters of the compound and/or prodrugs.

1. The term “admixing” is defined as mixing the two components, and any additional optional components, together. Depending upon the properties of the components to be admixed, there may or may not be a significant chemical or physical interaction between two or more components when they are mixed. For example, if one component is an acid, and the other component is a base, upon Admixing, the two components may, depending on the strength of the acids and bases, react to form a salt comprising the anion corresponding to the acid and the protonated cation corresponding to the base, or an equilibrium mixture of the original acids and bases, and their salts. In such cases, it will be understood by those of ordinary skill in the art that the resulting composition may be claimed in terms of the components known to be present after the admixing process, or alternatively may be claimed in terms of the components admixed in a product-by-process claim format, especially if the exact nature of the product resulting from the process of admixing the components is unknown or only poorly known or understood.

Disclosed herein are compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a number of different carrier molecules and protease inhibitors are disclosed and discussed, each and every combination and permutation of the carrier molecule and the protease inhibitor are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

The Inventions

In the many inter-related aspects of the inventions disclosed herein, the genera, subgenera, and species of compounds described hereinbelow, including their prodrugs and/or pharmaceutically acceptable salts, and their various pharmaceutical compositions and kits prepared thereof, can be used to treat or prevent muscle disorders, especially muscle disorders that result from the undesired catabolic activity of protease enzymes in muscle tissues, including serine, threonine and cysteine protease enzymes. In related aspects, the compounds described herein, including their prodrugs and/or pharmaceutically acceptable salts, and their various pharmaceutical compositions or kits thereof, can be used to treat or prevent muscle disorders that result from undesired proteolytic activity of calpain enzymes in muscle tissues, such as diseases or disorders exemplified by but not limited to a muscular dystrophy, a muscle wasting disease, cancer cachexia, cardiomyopathy, cardiac ischemia, denervation atrophy, or AIDS-related muscle wasting. In additional related aspects, the compounds described herein, including their prodrugs and/or pharmaceutically acceptable salts, and their various pharmaceutical compositions or kits thereof, can be used to treat or prevent Becker muscular dystrophy or Duchenn muscular dystrophy.

I. Compounds

The various compounds of the invention disclosed herein comprise a “carrier” molecule and/or the corresponding “carrier” functional group or residues that are either directly or indirectly bonded to another functional group or residue comprising one or more protease inhibitors.

The term “carrier molecule” as defined herein is any compound or functional group or residue thereof that can facilitate the delivery of the protease inhibitor into a muscle tissue. In one aspect, the carrier molecule can be any endogenous molecule. In an alternative embodiment, the carrier molecule can be a derivative of an endogenous compound.

In many embodiments, compounds comprising such carrier molecules or residues become selectively concentrated and/or actively or passively transported into muscle tissues, and in some embodiments into particular muscle tissues, including cardiac or skeletal muscle. For example, compounds comprising a carnitine or aminocarnitine residue can be become selectively concentrated and/or actively or passively transported into muscle tissues.

In many such aspects, the carrier molecules or residues of the present invention include the structures 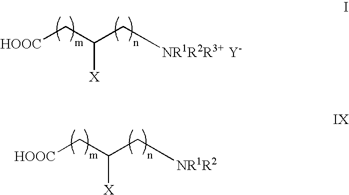
wherein each R¹, R², and R³ comprises, independently, hydrogen or a branched- or straight chain alkyl group,
X comprises OH or NHR⁶, or SH wherein R⁶ comprises hydrogen or a branched- or straight-chain alkyl group;
Y comprises a pharmaceutically-acceptable anion; and
m and n can be an integer from 1 to 10,
or the pharmaceutically-acceptable salt or ester thereof.

In some embodiments of the carrier molecules, R¹, R², and/or R³ are independently selected from the group consisting of hydrogen or C₁-C₆ alkyl groups, or substituted analogs of alkyl groups such as hydroxyalkyl groups or alkoxyalkyl groups. Alternatively, R¹, R², and/or R³ can independently selected from the group consisting of hydrogen or C₁-C₃ alkyl groups, such as methyl, ethyl, propyl, or isopropyl groups. In many embodiments, R¹, R², and/or R³ are all methyl groups.

As noted above, in some embodiments of the carrier molecules, m and n can be independently selected from integers between 1 and 10. In related aspects, m and n can be an integer from 1 to 10, 1 to 9, 1 to 8, 1 to 7, 1 to 6, 1 to 5, 1 to 4, or 1 to 3. In many embodiments, m and n are both the integer 1.

In many embodiments, the carrier molecule has the structure: 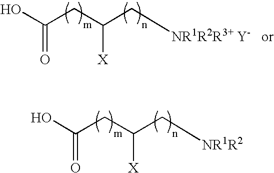
wherein each R¹, R², and R³ is independently selected from the group consisting of hydrogen or a branched- or straight chain alkyl group having 1 to 6 carbon atoms,
X is an O or NR⁶ group, wherein R⁶ is hydrogen or a branched- or straight-chain alkyl group having 1 to 6 carbon atoms;
m and n are independently selected from an integer from 1 to 10, and
Y is a pharmaceutically-acceptable anion.

One of ordinary skill in the art will understand that in the embodiments of the carrier molecules listed above, which can serve as analogs of carnitine and/or aminocarnitine, the X group comprising an OH or NHR⁶ radical is reacted with the protease inhibitor and/or the optional linker groups, to remove the hydrogen atom illustrated above and form the compounds of the invention wherein the X group is an O or NR⁶ radical that is directly or indirectly bonded to the protease inhibitor and/or the optional linker groups, as is illustrated below:

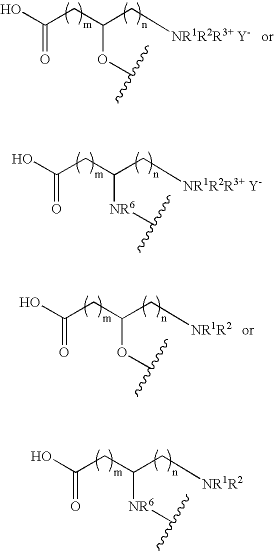

Moreover, one of ordinary skill in the art will recognize that compounds of the invention comprising the carrier molecules or residues can be present in the form of pharmaceutically acceptable salts, by pH dependent ionization of the carboxylic acid or protonation of the NR¹R² groups, or by formation of an ester prodrug of the carboxylic acid group of the carrier molecule, as is illustrated in the drawings below: 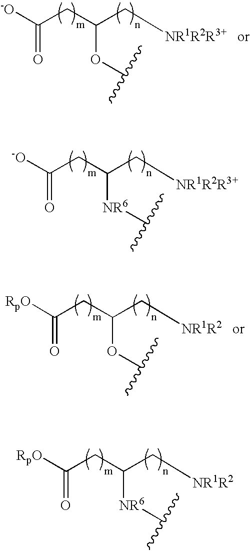
wherein the Rₚ group is an organic residue of 1 to 18 carbon atoms that forms the ester prodrug residues. The Rₚ could potentially be a wide variety of organic residues, such as phenyl or benzyl groups, hydroxyalkyl groups, or the like. In many embodiments, the Rₚ group is an alkyl group of 1 to 18, 1 to 12, 1 to 8, or 1 to 4 carbon atoms, or a methyl group.

In a related aspect, X is an OH group or oxygen atom, R¹, R², and R³ in formula I are each a methyl group, and m and n are 1. In this aspect, the carrier molecule can be referred to as carnitine, or a carnitine residue. In another aspect, X is NH₂, R¹, R², and R³ in formula I are each a methyl group, and m and n are 1. In this aspect, the carrier molecule can be referred to as aminocarnitine, or an aminocarnitine residue, as illustrated below:

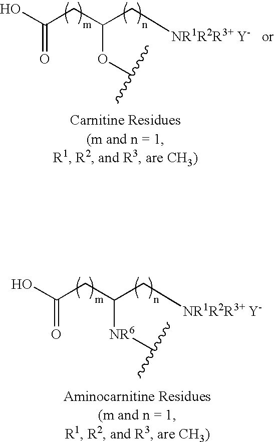

The carrier molecules useful herein possess a potentially optically active carbon atom bonded to the X group, and can be present in the form of the substantially pure L- or D-isomers, or a mixture thereof. The term “substantially pure” with respect to the L- or D-isomer refers to greater than 95%, greater than 97%, greater than 98%, greater than 99%, greater than 99.5%, or 100% of one isomer with respect to the other enantiomer. In many embodiments, the carrier molecules or residues are present as the substantially pure L isomers.

The term “protease inhibitor” is defined herein as any molecule that interacts with a protease and reversibly or irreversibly inhibits proteolytic activity of the protease. The term “protease inhibitor” also includes prodrug molecules that can be converted to a protease inhibitor upon administration to a subject. In some embodiments, the protease inhibitor can be an inhibitor of a serine, theonine, or cysteine protease. In related embodiments, the protease inhibitor can be a calpain inhibitor. In many embodiments, the protease inhibitor is a peptide aldehyde, a peptide boronate, or a vinyl sulfone. In another aspect, the protease inhibitor is a peptide having at least one aldehyde group or oxirane group. Examples of protease inhibitors include, but are not limited to, pepstatin, bestatin, Bowman-Birk inhibitor, chymostatin, bacitracin, lactacystin, clasto-lactacystin-β-lactone, ritonavir, saquinavir, indinavir, nelfinavir, amprenavir, calpastatin or a peptide fragment thereof, or the like.

In one aspect, the protease inhibitor is a calpain inhibitor. Examples of calpain inhibitors include, but are not limited to, leucyl-argininal, benzamidine derivatives, leupeptin, PhCH₂OCO-leucine-norvaline-CONH—CH₂-2-pyridyl, Ph₂CHCO-leucine-alpha-aminobutyric acid-CONH—CH₂-2-pyridyl, Ph₂CHCO-leucine-alpha-aminobutyric acid-CONH—(CH₂)₃₋₄-morpholinyl, PhCH₂OCO-leucine-alpha-aminobutyric acid-CONH—CH₂-2-pyridyl, and PhCH₂OCO-leucine-alpha-aminobutyric acid-CONH—CH₂—CH(OH)Ph.

In embodiments, the protease inhibitor is a residue containing one or more peptide-aldehyde protease inhibitors or one or more acetal prodrugs of a peptide aldehyde protease inhibitor. Many of such peptide-aldehyde protease inhibitors and acetal prodrugs of a peptide aldehyde protease inhibitors are also calpain inhibitors. Peptide-aldehyde protease inhibitor residues typically contain from 1 to 5 amino acid residues, but the carboxyl group of the last “amino acid” of the peptide chain has been replaced by an aldehyde group, or an acetal group, at the position that would normally be the carboxy-terminal position. The amino acids comprising such peptide aldehyde residues may comprise any amino acid residue comprising less than 18 carbon atoms, i.e. any compound containing less than eighteen carbon atoms comprising the residues of a carboxylic acid group and an amino group. In many embodiments the amino acid residues are an α-amino acid, and in many cases an amino acid selected from the 20 well known “standard” L-α-amino acids that commonly naturally occur in proteins and peptides, i.e. Alanine, Arginine, Asparagine, Aspartic acid, Cysteine, Glutamic acid, Glutamine, Glycine, Histidine, Isoleucine, Leucine, Lysine, Methionine, Phenylalanine, Proline, Serine, Threonine, Tryptophan, Tyrosine, and Valine, or any of a wide variety of analogs of the naturally occurring amino acids that are readily commercially available from a number of commercial chemical supply houses. The drawings below illustrate the chemical structures of peptide aldehydes and acetal prodrugs of peptide aldehydes comprising α-amino acids and a terminal aldehyde or acetal prodrug group:

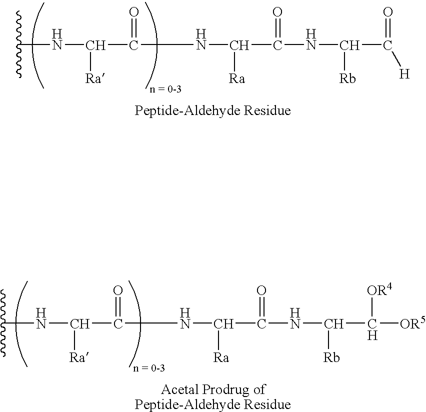

In the illustrated peptide aldehyde residues, Ra, Rb, and each Ra′ can be individually selected, and in many embodiments are selected from the side chains corresponding to the 20 naturally occurring amino acids. The “acetal” groups R⁴ and R⁵ are organic residues comprising 1 to 10 carbon atoms. In many embodiments, R⁴ and R⁵ can be, independently, a branched- or straight-chain alkyl group having from 1 to 20 carbon atoms or protecting group, or C(O)R¹⁰, where R¹⁰ can be independently a branched- or straight-chain alkyl group having from 1 to 20 carbon atoms, such as for example a methyl, ethyl, n-propyl, i-propyl, n-butyl, i-butyl, or t-buyl group, etc. Alternatively, R⁴ and R⁵ together form an alkylene residue that forms a five or six membered ring. In many embodiments, R⁴ and R⁵ are a branched- or straight chain alkyl group having one to three carbon atoms, such as methyl, ethyl, n-propyl, or i-propyl. In many preferred embodiments, R⁴ and R⁵ are both an ethyl group.

In some embodiments, the peptide aldehyde or acetal prodrugs of the peptide aldehyde is a dipeptide aldehyde or acetal prodrug thereof having one of the structures: 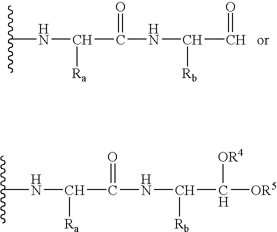
wherein R⁴ and R⁵ are a branched- or straight chain alkyl group having one to three carbon atoms, or wherein R⁴ and R⁵ together form an alkylene residue that forms a five or six membered ring.

In many embodiments, R⁴ and R⁵ are ethyl groups. Rₐ is hydrogen or a C₁-C₄ alkyl, and R_{b} is hydrogen, a C₁-C₄ straight or branched alkyl, or has one of the structures: 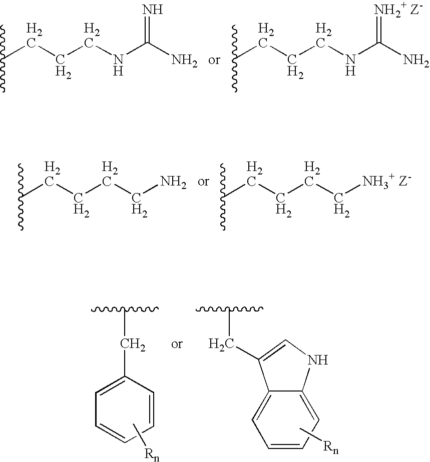
wherein Rₙ is hydrogen, hydroxyl, methyl, ethyl, methoxy, —NH₂, —NHCH₃, —N(CH₃)₂, —SH, or —SCH₃, and wherein Z⁻ is a pharmaceutically acceptable anion. In many such embodiments, Rₐ is an isopropyl, isobutyl, or 2-butyl group. In many such embodiments, R_{b} is an isopropyl, isobutyl, or 2-butyl group, or an optionally substituted benzyl or indanyl group having the structures shown above. In many such embodiments, Rₐ is a side chain corresponding to an arginine or lysine residue, i.e.: 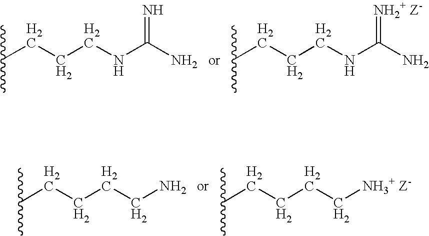
wherein those of skill in the art will understand that whether or not the side chain is present in its “neutral” or “cationic” form depends on the pH.

In some embodiments, the peptide aldehyde or acetal prodrug thereof is a leucyl argininal residue having the structure:

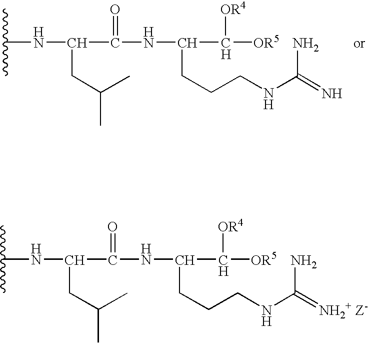

The carrier molecule and protease inhibitor can be directly or indirectly bonded to one another. When the carrier molecule and protease inhibitor are directly bonded to one another, a new covalent bond is formed between the carrier molecule and the protease inhibitor. For example, when X in formula I or IX is a hydroxyl group, the hydroxyl group can react with a COOH group or ester thereof present on the protease inhibitor to form a new ester linkage. In another embodiment, functional groups present on the carrier molecule and/or protease inhibitor can be chemically modified prior to direct or indirect bonding.

Alternatively, the carrier molecule and protease inhibitor can be indirectly bonded to one another with the use of a linker residue. A “linker molecule” is any compound that has at least one group that can form a covalent bond with the carrier molecule and at least one group that can form a covalent bond with the protease inhibitor. In one aspect, the linker group can have up to 25 carbon atoms. In related embodiments, the linker group can have from 1 to 18 carbon atoms, 1 to 12 carbon atoms, or 2 to 6 carbon atoms.

In one aspect, the linker group can be a polyether having two or more groups capable of reacting with the carrier molecule and protease inhibitor. For example, the polyether can have two or more hydroxyl groups, amino groups, carboxyl groups, or combinations thereof. It is contemplated that the polyether can be of varying molecular weight. Examples of linker molecules include, but are not limited to, □-aminocaproic acid, polyethyelene glycol, glutaraldehyde, and the like. It is contemplated that the linker molecule can be covalently attached to the carrier molecule, the protease inhibitor, or both prior to linking the carrier molecule to the protease inhibitor.

In some embodiments, the linker has the formula —C¹(O)(CH₂)ₒ(O)C²—, wherein o is an integer of from 1 to 10, 1 to 9, 1 to 8, 1 to 7, 1 to 6, or 1 to 5, 1 to 4, 1 to 3, or 2, wherein C¹ is covalently bonded to the carrier molecule or residue and C² is covalently bonded to the protease inhibitor or residue, or an acetal prodrug thereof.

In one embodiment, if the active compound has an aldehyde group, the aldehyde group can be converted to an acetal or hemiacetal using techniques known in the art, which can be cleaved under acidic conditions to produce the active form of the compound (i.e., reproduce the aldehyde group). Prodrug forms of the compounds described herein can also be more stable when compared to the active form, which also prolongs the shelf-life of the compound. It is contemplated that the carrier molecule, the protease inhibitor, or both can have groups that can be protected to convert the compound to the prodrug.

Any of the compounds described herein can be present in the form of the pharmaceutically-acceptable salt or ester thereof. In one aspect, pharmaceutically-acceptable salts are prepared by treating the free acid with an appropriate amount of a pharmaceutically-acceptable base. Representative pharmaceutically-acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

In another aspect, if the compound possesses a basic group, it can be protonated with an acid such as, for example, HCl, HBr, or H₂SO₄, to produce the cationic salt. For example, the techniques disclosed in U.S. Pat. No. 5,436,229 for producing the pharmaceutically acceptable sulfate salts of argininal aldehydes, which is incorporated by reference in its entirety, can be used herein. In the case of H₂SO₄, the corresponding sulfate (SO₄⁻²) or hydrogen sulfate (HSO₄⁻) salt can be produced depending upon the amount of acid that is employed. In one aspect, the reaction of the compound with the acid or base is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

Ester derivatives are typically prepared as precursors to the acid form of the compounds—as illustrated in the examples below—and accordingly can serve as prodrugs. Generally, these derivatives will be lower alkyl esters such as methyl, ethyl, and the like. Amide derivatives —(CO)NH₂, —(CO)NHR and —(CO)NR₂, where R is an alkyl group defined above, can be prepared by reaction of the carboxylic acid-containing compound with ammonia or a substituted amine.

In one aspect the compound is produced from a carrier molecule having the formula I that is indirectly linked to a protease inhibitor by a linker molecule. In one aspect, the compound is produced from carnitine or aminocarnitine, leucine or a derivative thereof, and arginine or a derivative thereof. In one aspect, the compound can be (1) carnitine or aminocarnitine covalently bonded to leucine or a derivative thereof through a linker such as, for example, glutaric acid or succinic acid, and (2) the leucine residue is covalently bonded to arginine or a derivative thereof. In another aspect, the compound has the formulae II or III: 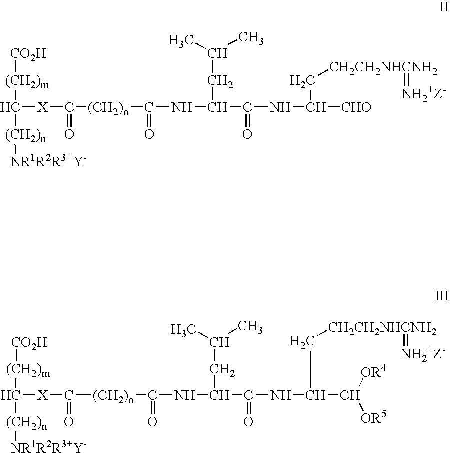
wherein R¹-R³, X, Y, m and n are defined as above, o is an integer from 1 to 10, and Z is a pharmaceutically-acceptable anion, and R⁴ and R⁵ can be, independently, a branched- or straight-chain alkyl group having from 1 to 20 carbon atoms or protecting group, or C(O)R¹⁰, where R¹⁰ can be independently a branched- or straight-chain alkyl group having from 1 to 20 carbon atoms. Alternatively, R⁴-R⁵ can be part of a ring. In one aspect, the compound has the formula II, wherein R¹, R², and R³ are methyl, m and n are 1, X is NH, and o is 1, 2, 3, 4, 5, 6, 7, 8, 9, 10. In another aspect, the compound has the formula III, wherein R¹, R², and R³ are methyl, R⁴ and R⁵ are butyl, m and n are 1, X is NH, and o is 1, 2, 3, 4, 5, 6, 7, 8, 9, 10. In another aspect, the compound has the formula III, wherein m and n are 1, R¹-R³ are methyl, R⁴ and R⁵ are each C(O)R¹⁰, X is NH, and o is 2. In a further aspect, the compound has the formula III, wherein the compound can be hydrolyzed at a pH of 1 to 3 at a temperature of 37° C.

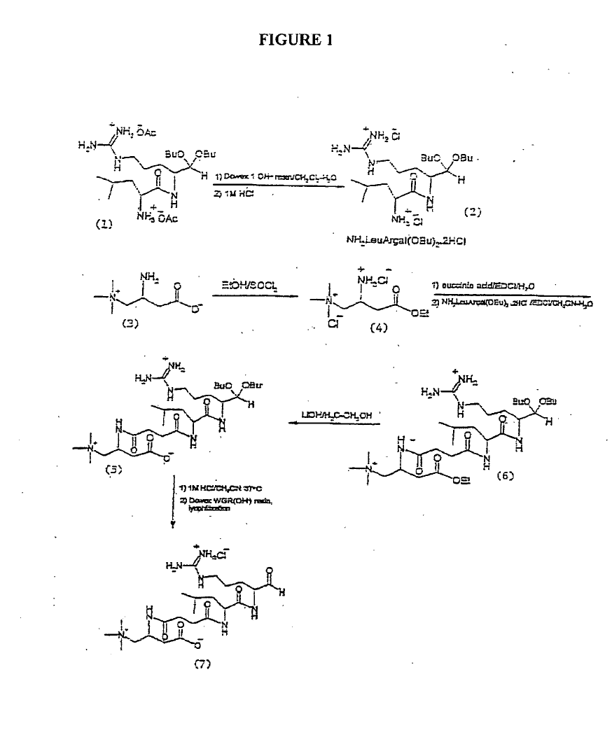

In another aspect, the compound has the formulae IV or V 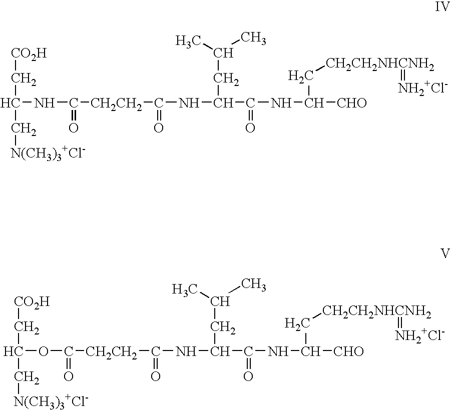
where compound IV has the chemical name aminocarnityl-succinyl-leucyl-argininal, which is also referred to herein as CLA. In one aspect, a synthetic scheme for producing compound IV is depicted in FIG. 1. Prodrugs of compounds IV and V are also contemplated. In one aspect, the prodrug can have the formulae VI and VII 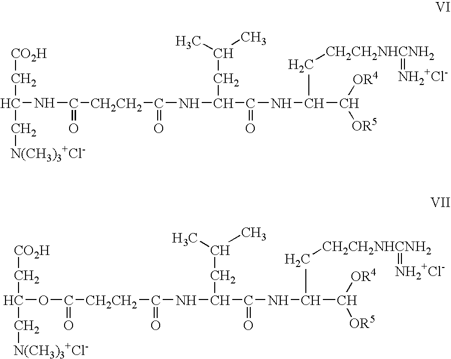
wherein R⁴ and R⁵ can be, independently, a branched- or straight-chain alkyl group having from 1 to 20 carbon atoms or protecting group, or C(O)R¹⁰, where R¹⁰ can be independently a branched- or straight-chain alkyl group having from 1 to 20 carbon atoms. In this aspect, formulae VI and VII are the diacetals and diesters of formulae IV and V, respectively. Alternatively, R⁴-R⁵ can be part of a ring. In this aspect, formulae VI and VII are cyclic acetals of formulae IV and V. In one aspect, R⁴ and R⁵ of formulae VI and VII can be, independently, methyl, ethyl, propyl, isopropyl, butyl, isobutyl, pentyl, isopentyl, or hexyl. In another aspect, R⁴ and R⁵ in formulae VI and VII are ethyl. In formulae II-VII, any pharmaceutically-acceptable salt or ester thereof is contemplated. In the case of formulae VI and VII, in one aspect, the acetal can be readily converted to the aldehyde by exposing the acetal to an acid. The synthetic techniques disclosed in U.S. Pat. Nos. 4,742,081; 4,866,040; 5,008,288; and 5,876,747, which are incorporated by reference for their teachings, can be used to produce the compounds and prodrugs in formulae II-VII, respectively.

In another aspect, the compound has the formula VIII: 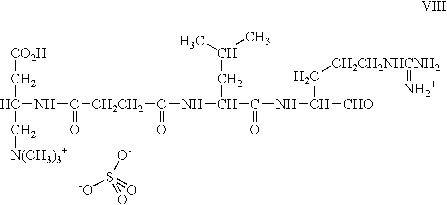
which is the sulfate salt of aminocarnityl-succinyl-leucyl-argininal. The compound having the formula VIII can also exist as the hydrogen sulfate salt (HSO₄⁻)

In many embodiments, when the protease inhibitor is leucyl-argininal in any of the formulae described above, the stereochemistry about the α-carbons of the two stereocenters of the leucyl and argininal residues of leucyl-argininal is L and L. In other aspects, the stereochemistry about the two stereocenters can be L, D; D, L; or D, D.

II. Synthetic Methods

Carnitine is widely commercially available. Aminocarnitine can be synthesized from 6-(dimethylaminomethyl)uracil as described by Jenkins, D. J. and Griffith, D. W. J. Biol. Chem. (1985) 260, 14,748-14,755, or by the methods described in U.S. Pat. No. 6,822,115. A very wide variety of methods for preparing peptides, including peptide aldehydes, are well known to those of ordinary skill in the art. A very wide variety of amino acids and peptides are available from well known commercial suppliers that include Bachem A G, of Bubendorf Switzerland, and Peptec Incorporated of Burlington Mass. Leupeptin is commercially available from Bachem A G.

Preparation of Aminocarnitine Analogs

The carnitine and aminocarnitine analog compounds of Formulas I and IX can be readily made by a variety of methods well known to those of ordinary skill in the art of organic synthesis. For example, methods for making substituted cyclic ketones of many ring sizes are known in the literature, and such compounds could be used as starting materials. As shown in the drawing below, such substituted ketones, if suitably protected, can be condensed with hydroxylamines to make the corresponding oxime compounds, and oxime compounds are well known to undergo Lewis acid catalyzed “Beckman Rearrangement” reactions to form the corresponding cyclic lactams, which could then be hydrolyzed to form the desired aminocarnitine analogs. 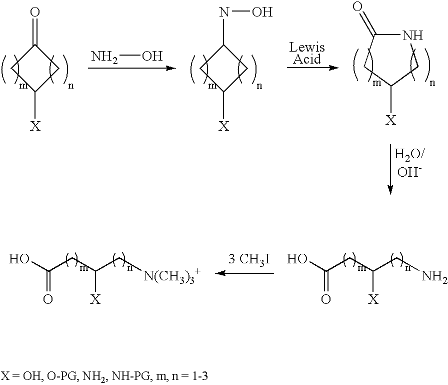
Preparation of Leucyl-Argininal Acetals
Leupeptin (a mixture of N-acetyl and N-propionyl leucylleucylarginal) is first converted to the hydrochloride chloride salt by passing it through a 1×8 column of a Dowex ion exchange resin in chloride form. The material is eluted with water and fractions are collected. Each fraction is tested for leupeptin by thin layer chromatography on silica gel using the upper phase of the solvent mixture butanol-acetic acid-water (4:1:5). Visualization is with iodine vapor. The fractions containing the leupeptin are combined and are evaporated to dryness on a Rotovap at room temperature.

The dry residue comprising leupetin hydrochloride is taken up in butanol and refluxed for 2 hours. This produces the dibutyl acetal of leupeptin, whose structure is shown below. 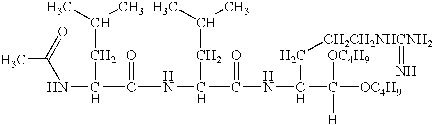
To the reaction product solution in a separatory funnel is added an equal volume of butanol and double the volume of water. After extraction, the upper butanol phase is removed and dried overnight with sodium sulfate. The solvent is then removed with a Rotovap.

The diethyl acetal of leupeptin can be produced in a similar manner by initial reflux of the leupeptin hydrochloride in ethanol rather than butanol, followed by vacuum evaporation of the ethanol, followed by butanol/water extraction and further purification as described above.

To purify the acetals of leupeptin, they are separated on a silica gel column using a gradient starting with chloroform and ending with 60% butanol/chloroform. The purified N-propionyl and N-acetyl forms are identified by thin layer chromatography as described above. The fractions are pooled and evaporated to dryness.
Cleavage of the N-propionyl and N-acetyl leucine residues from the leucyl argininal acetal residue is accomplished by hydrolysis in the presence of the enzyme thermolysin. This is done at pH 8, over a 72 hour incubation period. The progress of the hydrolysis reaction is followed by measuring the release of free amino groups by the TNBS reaction, to produce the acetals of leucyl argininal as shown below.

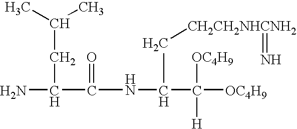

The leucylarginal dibutyl acetal is isolated by chromatography on a silica gel column as previously described. The fractions are identified by thin layer chromatography or other appropriate methods and the solvent is removed. The dry residue is stored in the freezer.

Linkage of Carnitine and Aminocarnitine Analogs to Protease Inhibitors

The hydroxyl group of carnitine and its analogs, and the amino group of aminocarnitine and its analogs can be directly or indirectly bonded to a host of protease inhibitors and linker groups to provide compounds within the scope of the invention. See for example U.S. Pat. Nos. 4,742,081, 4,866,040, 5,008,288, and 5,876,747, which are hereby incorporated by reference in their entirety for all purposes, including their disclosures of compounds and methods of synthesis.

The linkage of n-terminal peptide aldehyde residues, exemplified by leucyl-argininal acetals, or leucyl argininal itself, to the hydroxyl group of carnitine and its analogs, and the amino group of aminocarnitine and its analogs can be made by stepwise or simultaneous condensation with various difunctional linker groups, such as di-functional ketones or aldehydes such as glutaraldehyde, di-functional carboxylic acid equivalents such as glutaric acid, succinic acid, succinic anhydride, maleic anhydride, and the like, difunctional alkyl halides, etc., to form the linked compounds of the invention.

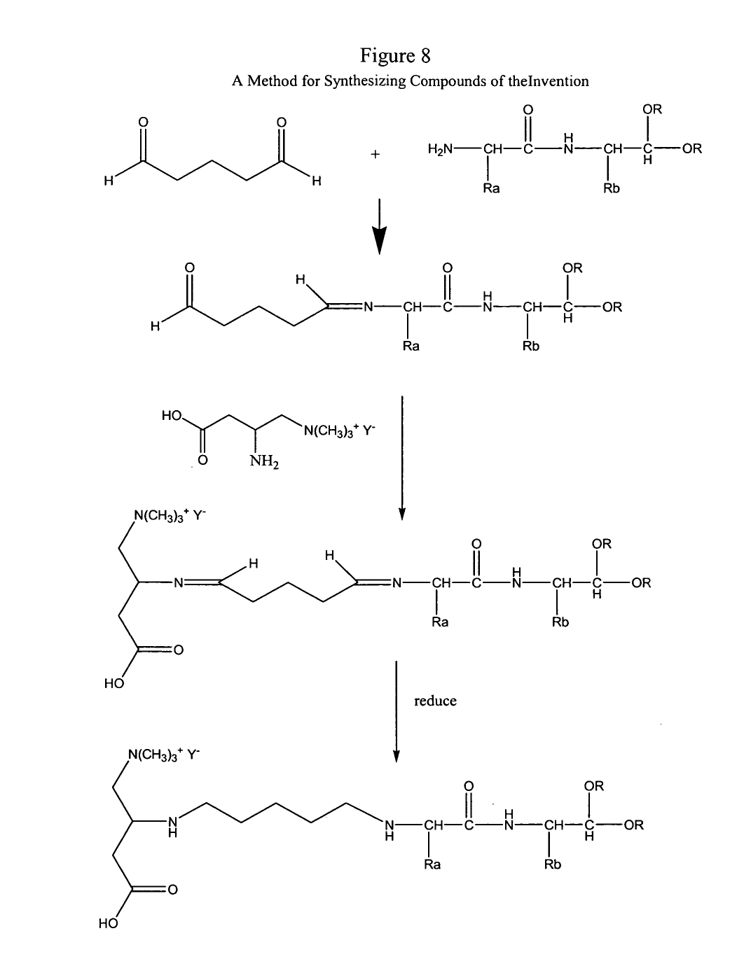

For example, an acetal of a peptide-aldehyde such as leucyl-argininal diethylacetal can be reacted with 1 molar equivalent of a bis-aldehyde such as glutaraldehyde to form a mono-imine, which would then be reacted with a molar equivalent of aminocarnitine or an analog thereof, to form a bis-imine compounds such as the compound shown below, whose imine groups can be reduced by any of a variety of methods known to those of ordinary skill in the art, such as catalytic hydrogenation or reduction with cyanoborohydride, to give an alkylene linked compound of the invention, as shown in FIG. 8.

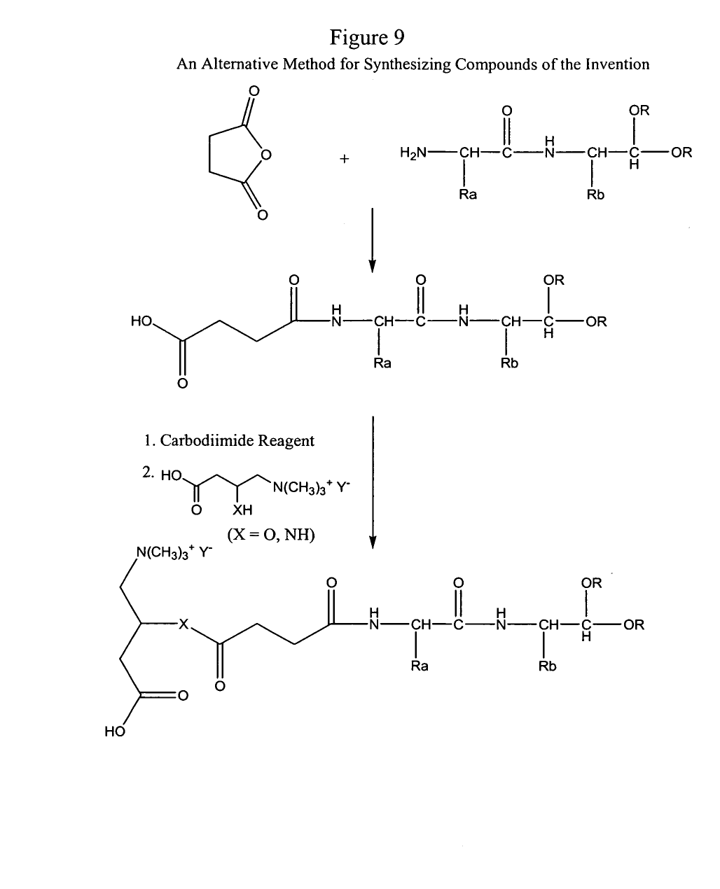

An alternative generic method of linking peptide aldehyde acetals to carnitine or aminocarnitine or their analogs is exemplified in FIG. 9. The peptide aldehyde acetal or an appropriately protected analog thereof can be condensed with an activated dicarboxylic acid or ester (illustrated in the Figure by succinic anhydride, but could be any dicarboxylic acid that had been activated by ring or ester formation, or by reaction with carbodiimide reagents such as 1-ethyl-3-(3-dimethylaminopropyl) carbodiimide) to bond the peptide aldehyde to the linker group terminated by a carboxylic acid group. The carboxylic acid group of the resulting intermediate compound could again be activated by reaction with a carbodiimide or similar reagent, and then condensed with a carnitine or aminocarnitine residue as is illustrated in a specific case by FIG. 9.

A specific procedure for the synthesis of CLA acetal (aminocamityl-succinyl-leucyl-argininal diethyl acetal, as a monoacetate salt) is provided in Example 13.

Accordingly in some embodiments, the invention relates to a method for synthesizing aminocarnityl-succinyl-leucyl-argininal acetal compounds comprising the steps of
a) coupling an alkyl ester of aminocarnitine with a benzyl ester of leucyl succinate to form an alkyl, benzyl ester of aminocarnityl-succinyl-leucine;
b) removing the benzyl group of the alkyl, benzyl ester of aminocarnityl-succinyl-leucine by catalytic hydrogenation to form an alkyl ester of aminocarnityl-succinyl-leucine;
c) coupling the alkyl ester of aminocarnityl-succinyl-leucine with a dialkyl acetal of an argininal compound having two oxycarbonyl protecting groups attached to the guanidine residue of the dialkyl acetal of argininal, to form an alkyl ester of aminocarnityl-succinyl-leuycl-argininal acetal;
d) removing the alkyl ester protecting group of the alkyl ester of the aminocarnityl-succinyl-leuycl-argininal acetal by saponification, to form an aminocarnityl-succinyl-leuycl-argininal acetal compound having oxycarbonyl protecting groups attached to the guanidine residue of the dialkyl acetal of argininal;
e) removing the oxycarbonyl protecting groups by catalytic hydrogenation, to form at least some of an aminocarnityl-succinyl-leucyl-argininal dialkyl acetal.

Further description of preferred embodiments of the above-described method of synthesis can be described by reference to Example 13.

Those of ordinary skill in the well developed arts of synthetic organic chemistry and/or synthetic peptide chemistry will, in light of the guidance and procedures provided herein and the incorporated references, readily be able to adapt and devise numerous alternative methods for synthesizing compounds within the scope of the inventions.

III. Pharmaceutical Compositions

Any of the carrier molecules or residues, linkers, and/or protease inhibitors described herein, and the compounds derived therefrom, can be employed in the form of a pharmaceutical composition, or used to prepare or manufacture pharmaceutical compositions or medicaments.

Any of the compounds described above, including prodrugs and active compounds produced by the kit, can be combined with at least one pharmaceutically-acceptable carrier to produce a pharmaceutical composition. The pharmaceutical compositions can be prepared using techniques known in the art. The composition can be prepared by admixing the compound with a pharmaceutically-acceptable carrier. Many pharmaceutically-acceptable carriers are known to those skilled in the art. These most typically would be standard carriers for administration to humans, including solutions such as sterile water, saline, and buffered solutions at physiological pH, which may optionally contain certain pharmaceutically acceptable solvents such as ethanol or dimethylsulfoxide. Many pharmaceutically acceptable solid carriers are also well known to those of ordinary skill, such as for example many mono-, di-, and polysaccharides such as sucrose, lactose, starches, pectins, and the like, as well as as semi-synthetic or synthetic polymer such as hydroxyalkyl celluloses, dextrans, polyacrylates, polyvinylpyrrolidones, and the like.

The pharmaceutical carrier(s) must be ‘acceptable’ in the sense of being compatible with the other ingredients of the composition and not overly deleterious to the recipient thereof.

The pharmaceutical compositions of the invention include sterile aqueous or non-aqueous solutions, suspensions, and emulsions. Examples of aqueous or non-aqueous carriers include water, alcoholic/aqueous solutions, emulsions or suspensions, including saline and buffered media. Parenteral vehicles, if needed for collateral use of the disclosed compositions and methods, include sodium chloride solution, Ringer's dextrose, dextrose and sodium chloride, lactated Ringer's, or fixed oils. Intravenous vehicles, if needed for collateral use of the disclosed compositions and methods, include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

The pharmaceutical compositions can, where appropriate, be conveniently presented in discrete unit dosage forms and can be prepared by any of the methods well known in the art of pharmacy. Such methods include the step of bringing into association the active compound with liquid carriers, solid matrices, semi-solid carriers, finely divided solid carriers or combination thereof, and then, if necessary, shaping the product into the desired delivery system.

Pharmaceutical compositions suitable for oral administration can be presented as discrete unit dosage forms such as hard or soft gelatin capsules, cachets or tablets each containing a predetermined amount of the active ingredient; as a powder or as granules; as a solution, a suspension or as an emulsion. The active ingredient can also be presented as a bolus, electuary or paste. Tablets and capsules for oral administration can contain conventional excipients such as binding agents, fillers, lubricants, disintegrants, or wetting agents. The tablets can be coated according to methods well known in the art., e.g., with enteric coatings.

Oral liquid preparations can be in the form of, for example, aqueous or oily suspensions, solutions, emulsions, syrups or elixirs, or can be presented as a dry product for constitution with water or other suitable vehicle before use. Such liquid preparations can contain conventional additives such as suspending agents, emulsifying agents, non-aqueous vehicles (which can include edible oils), or one or more preservative.

The compounds can also be formulated for parenteral administration (e.g., by injection, for example, bolus injection or continuous infusion) and can be presented in unit dose form in ampules, pre-filled syringes, small bolus infusion containers or in multi-does containers with an added preservative. The compositions can take such forms as suspensions, solutions, or emulsions in oily or aqueous vehicles, and can contain formulatory agents such as suspending, stabilizing and/or dispersing agents. Alternatively, the active ingredient can be in powder form, obtained by aseptic isolation of sterile solid or by lyophilization from solution, for constitution with a suitable vehicle, e.g., sterile, pyrogen-free water, before use.

Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents. The active ingredient can also be delivered via iontophoresis, e.g., as disclosed in U.S. Pat. No. 4,140,122, 4383,529, or 4,051,842; incorporated herein by reference.

Compositions suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described compositions can be adapted to provide sustained release of the active ingredient employed, e.g., by combination thereof with certain hydrophilic polymer matrices, e.g., comprising natural gels, synthetic polymer gels or mixtures thereof. The pharmaceutical compositions according to the invention can also contain other adjuvants such as flavorings, coloring, antimicrobial agents, or preservatives.

In view of the “Background” disclosures herein above, it has been known in the art that certain peptide aldehydes and analogs thereof can be employed in the active aldehyde form to prepare pharmaceutical compositions for inhibiting proteases and/or calpains in order to treat muscular diseases. Certain acetals of aldehydes, such as peptide aldehydes, have in some cases been previously been employed during the synthesis of those active aldehyde compounds. Nevertheless, the Inventors are unaware of any teachings or suggestions in the prior art that the acetals of aldehydes could be effectively used as prodrugs that can be directly administered to patients, or to prepare pharmaceutical compositions for direct administration to subjects, especially in the context of compounds useful as protease and/or calpain inhibitors.

It has been very unexpectedly discovered by the Inventors that the acetal prodrug forms of the protease inhibitor compounds of the invention disclosed herein can be effectively administered directly to subjects in the acetal form, both orally and parenterally, intraveneously and that unexpected advantages can result. Accordingly, some embodiments of the present inventions relate to a pharmaceutical composition for treating or preventing a muscle disorder comprising:
a) an acetal prodrug of a compound or a pharmaceutically acceptable salt or ester thereof, in an amount effective to treat or prevent a muscle disorder in a subject, wherein the compound comprises a carrier molecule and one or more protease inhibitors comprising an acetal of an aldehyde group, and
b) one or more pharmaceutically acceptable carriers;
   wherein the carrier molecule has the structure 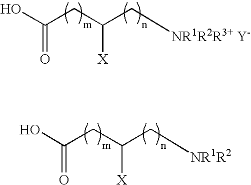
   wherein each R¹, R², and R³ is independently selected from the group consisting of hydrogen or a branched- or straight chain alkyl group having 1 to 6 carbon atoms,
   X is an O or NR⁶ group, wherein R⁶ is hydrogen or a branched- or straight-chain alkyl group having 1 to 6 carbon atoms;
   m and n are independently selected from an integer from 1 to 10, and Y is a pharmaceutically-acceptable anion; and
   wherein X is directly bonded to the one or more protease inhibitors or X is indirectly bonded to the one or more protease inhibitors through a linker comprising up to 25 carbon atoms.

Again, it is hereby specifically contemplated that any of the carrier molecules or residues, linkers, protease inhibitors, or the acetal prodrug compounds formed therefrom, or the relevant description of the subgenuses or other variables relating to genera, subgenera, or species of compounds, or their pharmaceutically acceptable salts or prodrugs thereof can be employed to make the pharmaceutical compositions of the invention.

The acetal prodrugs employed in the present pharmaceutical compositions can, in some cases of oral administration, undergo significant hydrolysis to the free aldehyde form while passing through the acidic conditions of the stomach. Nevertheless, much of any remaining acetal prodrug form of the active compound can also be directly absorbed by the digestive tract into systemic circulation. Moreover, Applicants have very unexpectedly discovered evidence as exemplified by the examples herein, that the acetal prodrug forms of the aldehyde compounds, once initially absorbed in prodrug form, conconcentrate in muscular tissues as desired, and moreover are subsequently hydrolyzed in-vivo to the active free aldehyde form of the compound that serves as a protease and/or calpain inhibitor.

IV. Kits

Described herein are kits useful for treating or preventing a muscle disorder. In one aspect, described herein is a kit for treating or preventing a muscle disorder in a subject comprising:
a. an effective amount of a prodrug of a compound, wherein the compound comprises a carrier molecule and a protease inhibitor, wherein the carrier molecule is directly or indirectly bonded to one or more protease inhibitors, and
b. an activator, wherein the activator converts the prodrug to the active form of the compound.

It is contemplated that prodrugs, especially the acetal prodrugs of any of the compounds described above can advantageously be administered in kit form. In some embodiments, the inventions relate to kit for treating or preventing a muscle disorder comprising:
a. an acetal prodrug of a compound, or a pharmaceutically acceptable salt or ester thereof, in an amount effective to treat or prevent a muscle disorder in a subject, wherein the compound comprises a carrier molecule and one or more protease inhibitors comprising an acetal of an aldehyde group, and
b. an activator for converting the acetal prodrug of the compound to the aldehyde form of the compound;
   wherein the carrier molecule has the structure 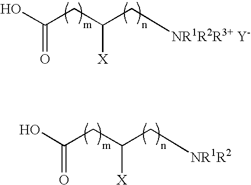
   wherein each R¹, R², and R³ is independently selected from the group consisting of hydrogen or a branched- or straight chain alkyl group having 1 to 6 carbon atoms,
   X is an O or NR⁶ group, wherein R⁶ is hydrogen or a branched- or straight-chain alkyl group having 1 to 6 carbon atoms;
   m and n are independently selected from an integer from 1 to 10, and
   Y is a pharmaceutically-acceptable anion; and
   wherein X is directly bonded to the one or more protease inhibitors or X is indirectly bonded to the one or more protease inhibitors through a linker comprising up to 25 carbon atoms.

The activator used in the kit is a compound or mixture of compounds that, in the presence of water, converts and/or hydrolyzes the prodrug to the active form of the compound, and in the case of the acetal prodrugs to the free aldehyde form of the compound. The term “active compound” as defined herein is the compound that induces or potentiates a desired response. For example, the compounds depicted in formula III are acetals. Treatment of the acetal III with an acid (typically an aqueous acid) produces compounds having the formula II, which is an aldehyde. Not wishing to be bound by theory, it is believed that the aldehyde group in formula II makes compounds in formula II the active compound that can treat or prevent a muscle disorder in a subject. The selection of the activator will vary depending upon the prodrug (i.e., the nature of the protecting group). Examples of activators include, but are not limited to, inorganic acids such as, for example, hydrochloric acid, sulfuric acid, nitric acid, phosphoric acid, organic acids such as, for example, maleic, oxalic, citric, tartaric, and acetic acids. In addition, activators may include substances that act as catalysts for activation such as, for example, enzymes, metals, salts, polymers, detergents, and zeolites. Examples of enzymes useful herein include hydrolases, esterases, glycosidases, glycanases, proteases, lipid-metabolizing enzymes, oxidases, and cytochrome P450 enzymes.

It has been discovered that such kits comprising acetal prodrugs can be particularly effective method for synthesis, storage, and administration of the peptide aldehyde compounds of the invention, which are well known in the art to be undesirably chemically and stereochemically unstable during both synthesis, compounding, and administration. In particular, it is well known that the α-carbon of the terminal amino-aldehyde can readily enolize at significant rates at room temperature, with resulting racemization of the stereocenter of the P1 residue. In view of the often higher biological activity of compounds comprising amino acid residues in the natural L optical configurations, such racemization of the free aldehydes is highly undesirable. The acetal prodrug forms of the peptide-aldehydes described herein are typically much more chemically and stereochemically stable during manufacture, and are typically much more easily formulated and stored in the form of a kit which can have much better shelf life than pharmaceutical compositions and/or kits that comprise the free aldehyde form of the same drug. Nevertheless, when rapid absorption of the aldehyde form of the drug is clinically desirable, the kit comprising the acetal prodrug and an activator can be employed to generate desired percentages, or quantitive conversion of the free aldehyde form of the drug, which is then administered to the patient.

Depending upon the intended clinical use of the kit, the prodrug, activator, and neutralizing agent can exist as solids or solutions. For example, it is contemplated that the prodrug be in a solid or aqueous solution and the activator be in aqueous solution. Alternatively, it is possible that the prodrug and the activator are both in solid form together or separately. In this aspect, water alone or in combination with other components can be added to the solid mixture. For example, sterile water, saline, and buffered solutions at physiological pH can be used to store the prodrug or activate the prodrug with the activator.

The prodrug and activator can be admixed in any order. The duration of the admixing can vary depending upon the prodrug and activator selected as well as the relative amount of activator to prodrug. Depending upon the selection of the activator and the prodrug, it may be desirable to heat the prodrug and the activator upon mixing in order to expedite the conversion of the prodrug to the active compound. In one aspect, the kit further comprises a heating device. In one aspect, the heating device can be a Peltier block modified to hold a vial containing a mixture of the prodrug and activator. It is also contemplated that the kit can comprise a cooling device to cool the solution of the active compound If the solution was heated by the heating device.

It should be understood that it is not always necessary that all or almost all of the prodrug form of the compounds be converted to the parent active compound, but in some embodiments, the duration of admixing is sufficient to convert the prodrug substantially (i.e., greater than 80%, greater than 85%, greater than 90%, greater than 95%, or greater than 97, 98, or 99%) or completely to the active compound. Once the activator and the prodrug are admixed to produce the active compound, the active compound can be administered to the subject by a variety of techniques. Methods for delivering the compounds described herein including the active compound produced from the kits will be described below.

Depending upon the activator that is used, a neutralizing agent can be an optional component of the kit. The neutralizing agent is any compound or mixture of compounds that renders the activating agent safe for consumption or administration to a subject after the prodrug has been converted to the active compound. For example, the neutralizing agent can render the activator inactive after the prodrug has been converted to the active compound or, in the alternative, convert the activator to a compound that is not harmful to a subject. The neutralizing agent will vary depending upon the selection of the activator. For example, when the activator is an acid, the neutralizing agent can be a strong inorganic base such as sodium hydroxide or a weak base such as dibasic sodium phosphate. In another aspect, once the activator has converted the prodrug to the active compound, the mixture can be diluted in a beverage to neutralize any remaining activator.

In further aspects of the kits of the inventions, after the acetal prodrug and activator have formed the aldehyde form of the drug, a neutralizing base or buffering agent can be added to reduce the acidity of the activated drug prior to administration. In another aspect, any of the compounds depicted in formulae III, VI, and VII can be used as the prodrug in the kit.

For example, in preferred embodiments of the invention, kits for orally administering the compounds of the invention can be prepared. In one non-limiting example, such a kit can comprise three capped vials, the first vial comprising about 500 mg of L-aminocamityl-succinyl-leucyl-argininal diethyl acetal, acetate salt (CLA Acetal), the second vial or container comprising excess volumes of 0.5 M Hydrochloric Acid USP, and a third vial comprising pharmaceutically acceptable aqueous solutions, such as aqueous solutions comprising well known sweeteners such Ora-Sweet SF™ Sweetener. About 4.3 mL 0.5M Hydrochloric Acid USP is added to the first vial comprising the CLA Acetal, then the capped vial is immersed in a water bath at 60° C. for 60 minutes, to quantitatively hydrolyze the CLA acetal to the parent CLA aldehyde. Excess heating should be avoided to avoid degradation of the CLA aldehyde solution so produced, then 45 milliliters of aqueous Ora-Sweet solution added, and the pH checked and adjusted with additional HCl solution to be above pH 3. The resulting solution, suitable for oral administration to human patients, should be stored in a refrigerator prior to administration to the patient at a concentration from about 0.5 to about 50 mg of CLA per kilogram patient weight per day.

In some embodiments the invention relates to a method for using the kits of the invention for treating or preventing a muscle disorder in a subject, comprising (a) admixing the acetal prodrug of the compound and the activator, to produce at least some of the aldehyde form of the compound and (b) administering to the subject the aldehyde form of the compound in an amount effective to treat or prevent the muscle disorder.

In certain preferred aspects of the kits of the invention, the acetal prodrug has the formula XI: 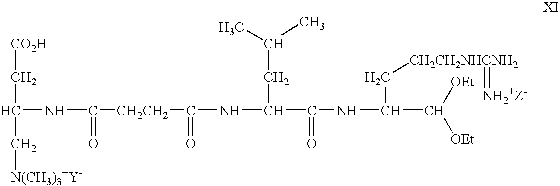
and the activator is HCl.
V. Methods of Use

Delivery

As used throughout, administration of any of the compounds or their pharmaceutically acceptable salts, including the prodrugs and the active compounds produce by the kits described herein, can occur in conjunction with other therapeutic agents. Thus, the compound can be administered alone or in combination with one or more additional therapeutic or prophylactic agents. For example, a subject can be treated with a compound alone, or in combination with chemotherapeutic agents, antibodies, antivirals, steroidal and non-steroidal anti-inflammatories such as prednisone, corticosteroids, conventional immunotherapeutic agents, cytokines, chemokines, and/or growth factors, as well as pharmaceutically acceptable carriers, diluents, additives, and the like. Combinations of the compounds of the invention and other agents may be administered either concomitantly (e.g., as an admixture), separately but simultaneously (e.g., via separate intravenous lines into the same subject), or sequentially (e.g., one of the compounds or agents is given first followed by the second). Thus, the term “combination” or “combined” is used to refer to either concomitant, simultaneous, or sequential administration of two or more agents.

The compounds, which include the prodrugs and the active compounds produced by the kits described herein, can be administered in a number of ways depending on whether local or systemic treatment is desired, and on the area to be treated. Administration may be topically (including opthalmically, vaginally, rectally, intranasally), orally, by inhalation, or parenterally, for example by intravenous drip, subcutaneous, intraperitoneal or intramuscular injection. The disclosed compounds can be administered intravenously, intraperitoneally, intramuscularly, subcutaneously, intracavity, transdermally, intratracheally, extracorporeally, sublingually, rectally, or topically (e.g., topical intranasal administration or administration by inhalant). As used herein, “topical intranasal administration” means delivery of the compositions into the nose and nasal passages through one or both of the nares and can comprise delivery by a spraying mechanism. The latter can be effective when a large number of subjects are to be treated simultaneously. Administration of the compositions by inhalant can be through the nose or mouth via delivery by a spraying mechanism or droplet mechanism. Delivery can also be directly to any area of the respiratory system (e.g., lungs) via intubation.

In some embodiments, when the compound is a prodrug, the compound can be administered orally, so that after digestion, the acidic conditions present in the stomach can convert the prodrug to the active form. For example, a compound having the formula VI, which is an acetal, can in some cases be administered orally and converted to the corresponding aldehyde IV by cleavage of the acetal group in vivo.

Parenteral administration of the compound, if used, is generally characterized by injection. Injectables can be prepared in conventional forms, either as liquid solutions or suspensions, solid forms suitable for solution or suspension in liquid prior to injection, or as emulsions. Alternatively, parenteral administration can involve the use of a slow release or sustained release system such that a constant dosage is maintained.

The exact amount of the compounds described herein required will vary from subject to subject, depending on the species, age, weight and general condition of the subject, the severity of the disorder being treated, the particular muscle tissue to be targeted, its mode of administration and the like. Thus, it is not possible to specify an exact amount for every compound. However, an appropriate amount can be determined by one of ordinary skill in the art using only routine experimentation given the teachings herein. The time at which the compounds can be administered will also vary depending upon the subject, the disorder, mode of administration, etc. The compound can be administered to the subject prior to the onset of the muscle disorder or during a time when the subject is experiencing symptoms of the muscle disorder. The compound can be administered over several weeks or months at varying intervals depending upon the subject and disorder to be treated. It is also contemplated that the compounds described herein can be administered daily in single or multiple doses.

The present compounds are generally administered in a therapeutically effective amount. That is, an amount that is sufficient to achieve the desired therapeutic result or to have an effect on undesired symptoms, but is generally insufficient to cause adverse side affects. For example, the compounds described herein can be administered in a therapeutically effective amount at a dosage from 0.01 mg/kg to 1,000 mg/kg, or preferably about 0.5 to about 50 50 mg/kg/day. In another aspect, the lower endpoint of the dosage is 0.01, 0.05, 0.10, 0.20, 0.30, 0.40, 0.50, 0.60, 0.70, 0.80, 0.90, 1.0, 2.0, 3.0, 4.0, 5.0, 6.0, 7.0, 8.0, 9.0, 10.0, 15.0, 20.0, 25.0, 30.0, 35.0, 40.0, 45.0, 50.0, 60.0, 70.0, 80.0, 90.0, 100.0, 150.0, 200.0, 250.0, 300.0, 350.0, 400.0, 450.0, 500.0, 600.0, 700.0, 800.0, or 900.0 mg/kg, and the upper endpoint of the dosage is 1.0, 2.0, 3.0, 4.0. 5.0, 6.0, 7.0, 8.0, 9.0, 10.0, 15.0, 20.0, 25.0, 30.0, 40.0, 50.0, 60.0, 70.0, 80.0, 90.0, 100.0, 150.0, 200.0, 250.0, 300.0, 350.0, 400.0, 450.0, 500.0, 550.0, 600.0, 650.0, 700.0, 750.0, 800.0, 850.0, 900.0, 950.0, or 1,000.0 mg/kg, where any lower endpoint can be used with any upper endpoint, where the lower endpoint is less than the upper endpoint. In many embodiments of the inventions, the aldehyde or acetal prodrug form of the compound, or their pharmaceutically acceptable salts are administered to the subject in an amount of at least about 0.1, or alternatively at least about 0.5, 1, 2, 3, 4, 5, 10, 20, 50, 100 mg/kg/day. In related embodiments, the dosage is from 1 to 10 mg/kg or 1 to 5 mg/kg, administered at intervals determined as suitable by an attending physician.

Molecules intended for pharmaceutical delivery may be formulated in a pharmaceutical composition. Pharmaceutical compositions may include carriers, sweetners, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally).

It will be appreciated that the actual preferred amounts of compound in a specified case will vary according to the specific compound being utilized, the particular compositions formulated, the mode of application, and the particular situs and mammal being treated. Dosages for a given host can be determined using conventional considerations, e.g. by customary comparison of the differential activities of the subject compounds and of a known agent, e.g., by means of an appropriate conventional pharmacological protocol. Physicians and formulators, skilled in the art of determining doses of pharmaceutical compounds, will have no problems determining dose according to standard recommendations (Physicians Desk Reference, Barnhart Publishing (2005).

In another aspect, any of the compounds described herein can be administered to a subject with the use of a liposome. In one aspect, the carrier molecule and/or protease inhibitor of the compound can be covalently attached to the liposome by reacting the carrier molecule and/or protease inhibitor with the fatty acid used to produce the liposome. In another aspect, the compounds described herein can be enclosed within the inner volume of the liposome and not covalently attached to the liposome. Examples of liposomes useful herein include, but are not limited to, conventional liposomes, long-circulating liposomes, immunoliposomes, and cationic liposomes. The methods disclosed in U.S. Pat. Nos. 4,866,040; 5,008,288; and 5,876,747, which are incorporated by reference, can be used to produce liposome formulations with the compounds described herein. In another aspect, the compounds described herein can be delivered to a subject via micelles, nanoparticles, microspheres, and lipoproteins.

Therapeutic Uses

In some aspects of the invention, disclosed are methods for preventing or treating a muscle disorder in a subject, which involves administering an effective amount of any of the compounds, pharmaceutical compositions, or kits described herein, including prodrugs and active compounds produced by the kits, to a subject in need of such treatment or prevention or diagnosed as having a muscular disease. Examples of muscle disorders that can be treated or prevented by the methods described herein include, but are not limited to, muscular dystrophies (e.g., DMD, BMD, LGMD, FSHMD); muscle wasting caused from, for example, cancer, denervation atrophy, AIDS, diabetes, malnutrition, or disuse; and cardiovascular disorders such as, for example, cardiomypathies and cardiac ischemia. All of these diseases are believed to be a result, at least in part, of undesirable proteolytic activity of protease enzymes.

In many embodiments of the invention, the compounds described herein can inhibit Ca-activated proteases (i.e. calpains) when an effective amount of compound is administered to a subject. In some aspects of the invention, the aldehyde and acetal prodrugs and/or their pharmaceutically acceptable salts and/or pharmaceutical compositions thereof, and active compounds produced by use of the kits described herein can be used to inhibit Ca-activated proteases.

In one aspect, the compounds described herein can treat muscular dystrophy in a subject. Muscular dystrophy (MD) refers to a group of genetic diseases characterized by progressive weakness and degeneration of the skeletal or voluntary muscles which control movement. The muscles of the heart and some other involuntary muscles are also affected in some forms of MD, and a few forms involve other organs as well. The major forms of MD include myotonic, Duchenne (DMD), Becker (BMD), limb-girdle (LGMD), and other forms. DMD is the most common form of MD affecting children. MD can affect people of all ages. Although some forms first become apparent in infancy or childhood, others may not appear until middle age or later. Administration of high doses of steroids such as Prednisone can slow the progression of some symptoms of DMD, there is no truly effective therapy for this horrible disease, which is essentially uniformly fatal. In another aspect, when a prodrug is administered to a subject, the prodrug can diffuses into muscle cells or preferably is taken up into muscle cells by an active or passive transport system (especially the well known biological tranporters of carnitine and its fatty acid derivatives, and converted to the active form of the compound. In this aspect, this is particularly useful because the prodrug does not get converted to the active compound until it reaches the desired target, which is muscle cells and tissue. In another aspect, the active compounds produced by the kits described herein can be used to treat muscular dystrophy.

Accordingly, some embodiments of the invention relate to a method for treating or preventing Duchene's muscular dystrophy in a mammal, including humans, comprising administering to the mammal one or more of the aldehyde or acetal prodrug compounds of the invention disclosed herein, or their pharmaceutically acceptable salts or pharmaceutical compositions, in an amount effective to treat or prevent Duchene's muscular dystrophy in the mammal. One embodiment of the invention relates to a method for treating or preventing Duchene's muscular dystrophy in a mammal, comprising administering to the mammal one or more aldehyde compounds, or pharmaceutically acceptable salts thereof, in an amount effective to treat or prevent Duchene's muscular dystrophy in the mammal, wherein the aldehyde compound has the structure 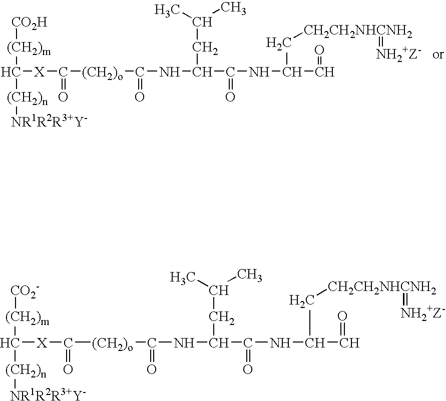
wherein each R¹-R³ is, independently, hydrogen or a branched- or straight chain alkyl group having 1 to 6 carbon atoms,
X is O or NR⁶, wherein R⁶ comprises hydrogen or a branched- or straight-chain alkyl group having 1 to 6 carbon atoms;
Y and Z are pharmaceutically-acceptable anions; and
m, n, and o can be an integer from 1 to 10,
or the pharmaceutically-acceptable salt or ester thereof.

In such embodiments, R¹-R³ are preferably methyl groups, m and n are 1, o is 2, and X is NH, and Y— and Z- are preferably chloride, acetate, bisulfate, sulfate, biphosphate, or succinate.

In additional related aspects, the compounds described herein can, when applied to muscle tissues afflicted with the muscle disorders disclosed herein, help to increase, the myofiber diameter of muscle tissue when the muscle tissue is contacted with an effective amount of the compound. In an additional related aspect, the compounds described herein can slow or prevent the further reduction of myofiber diameter or breakdown of myofibers when the muscle tissue is contacted with an effective amount of the compound. Alternatively, in another release aspect, the compounds described herein can increase the weight of existing muscle tissue when the muscle tissue is contacted with an effective amount of compound. In these aspects, the muscle tissue can be contacted in vitro, in vivo, or ex vivo. In one aspect, the prodrugs and active compounds produced by the kits described herein can be used to increase the myofiber diameter of muscle tissue.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1

### MDX Mouse Studies

It is well documented that as, observed in DMD and BMD, dystrophin is dysfunctional/absent in the murine MDX model of muscular dystrophy. In this mouse model for DMD/BMD, significantly elevated levels of intracellular calcium mediate an elevated rate of protein degradation in muscle cells. A series of studies to test the ability of the calpain inhibitor, leupeptin, to delay and/or prevent features of calcium-induced damage to dystrophic myofibers was initiated. The results are presented in Tables 1 and 2. Control C57BL/10SNJ mice were treated with intramuscular injections of normal saline. The MDX mice were treated intramuscularly with either normal saline or 12 mg/kg leupeptin or 18 mg/kg leupeptin twice daily. All injections were delivered in the hind-limbs in various locations to avoid muscle damage due to repeated injections at the same location. The gastrocnemius, soleus, anterior tibialis, and diaphragm muscles were examined in all animals. Table 1 shows the results of this analysis.

TABLE 1
Myofiber diameter (μM ± SD)

| | | | Anterior | |
|---|---|---|---|---|
| | Gastrocnemius | Soleus | tibialis | Diaphragm |
| C57BL/10 SNJ | 37.9 ± 3.8 | 20.3 ± 2.2 | 28.7 ± 1.8 | 22.1 ± 2.0 |
| mdx | 20.9 ± 0.9 | 19.2 ± 2.8 | 24.4 ± 1.5 | 16.4 ± 1.7 |
| mdx 12 mg/kg | 32.9 ± 2.6 | 24.7 ± 1.8 | 37.5 ± 2.8 | 21.8 ± 0.9 |
| leupeptin | | | | |
| mdx 18 mg/kg | 34.0 ± 2.7 | 25.5 ± 2.3 | 38.3 ± 1.3 | 21.7 ± 1.8 |
| leupeptin | | | | |

Comparison of mdx (untreated) muscles to 12 mg/kg and 18 mg/kg leupeptin treatment was significant, p < 0.01, for the gastrocnemius, anterior tibialis, and diaphragm muscles. Significance was at p < 0.05 in the soleus muscle (Student's two-tail t-test).

As shown above in Table 1, both doses of leupeptin, increased myofiber diameters in MDX leupeptin-treated muscles when compared with saline-treated control MDX muscles. Not wishing to be bound by theory, it is believed that leupeptin prevents a decrease in myofiber size rather than increasing an already degenerated myofiber. Further, in comparison to untreated MDX muscle in the gastrocnemius, treatment with 12 mg/kg leupeptin induced a 37% increase in myofiber diameter and, at 18 mg/kg, a 39% increase in myofiber diameter. In the soleus muscle, 12 mg/kg induced a 22% and 18 mg/kg a 25% increase in diameter. In the anterior tibialis, 12 mg/kg induced a 35% increase and, at 18 mg/kg, a 36% increase, demonstrating large increases in diameter. In the diaphragm, treatment at both 12 mg/kg and 18 mg/kg induced a 25% increase in myofiber diameter.

C57BL/1OSNJ control mice showed nornal histology. The histologic appearance of untreated MDX myofibers was characterized by evidence of myofiber degeneration consistent with other reports. Electron microscopy confirmed light-microscopic findings of larger myofibers with features of normal ultra structure after leupeptin treatment at both doses compared with saline-treated MDX muscle.

To further examine whether the beneficial/protective action of leupeptin was associated with a change in calpain activity, the activity of the enzyme was assessed in the muscle various muscles taken from the control, and MDX rats±treatment with leupeptin. As shown in Table 2, in the muscle taken from leupeptin-treated MDX mice, calpain activities decreased substantially in response to leupeptin treatment.

TABLE 2
Calpain activities of muscle samples^{a}

| | | | Anterior | |
|---|---|---|---|---|
| | Gastrocnemius | Soleus | tibialis | Diaphragm |
| C57BL/10 SNJ | 56 ± 5 | 123 ± 9 | 75 ± 7 | 22 ± 5 |
| mdx | 168 ± 11 | 335 ± 21 | 311 ± 17 | 58 ± 4 |
| 12 mg/kg | 27 ± 7 | 187 ± 16 | 155 ± 16 | 26 ± 4 |
| 18 mg/kg | 67 ± 8 | 102 ± 9 | 71 ± 11 | 17 ± 5 |

^{a}Activities based on cpm/200-μg protein; average of 3 determinations per sample; calpain assay described in Methods.
Comparison of mdx (untreated) muscles to 12-mg/kg and 18-mg/kg leupeptin treatment was significant, p < 0.01 for the gastrocnemius; p < 0.05 for the soleus (12 mg), p < 0.01 (18 mg); p < 0.05 for the anterior tibialis; and p < 0.05 for the diaphragm (Student's two-tail t-test).

In comparing the results of leupeptin-treated to untreated MDX muscles, in the gastrocnemius, at 12 mg/kg, there was an 84% decrease in calpain activity and, at 18 mg/kg, a 60% decrease. In the soleus muscle at 12 mg/kg leupeptin, there was a 44% decrease and, at 18 mg/kg leupeptin a 70% decrease in calpain activity. In the anterior tibialis at 12 mg/kg leupeptin, there was a 50% decrease, and a 77% decrease in calpain activity at 18 mg/kg of treatment. In the diaphragm muscle at 12 mg/kg, there was a 51% decrease, and a striking 71% decrease in activity at 18 mg/kg of leupeptin treatment.

This study demonstrates that leupeptin can inhibit muscle degeneration in this dystrophin deficient murine MDX model. In fact, the increase in myofiber diameter actually exceeded the normal size in treatments at both doses in both soleus and tibialis.

### Example 2

### Monkey Studies

### First Set of Monkey Studies: Intramuscular Injection of Leupeptin

In order to test the hypothesis that in vivo inhibition of calpains by leupeptin can facilitate neuromuscular recovery by inhibiting denervation atrophy of the skeletal-muscle target after complete peripheral nerve transection and delayed nerve repair (7), a monkey study (Cebus apella) was undertaken. This median-nerve model was chosen to approximate human anatomic and regenerative aspects.

Toxicology: There were no significant differences in all mean values between treated and control animals. Complete blood counts and clotting times after intramuscular leupeptin administration at 18 mg/kg, twice daily for 24 weeks, were not affected in treated animals, when compared to controls. In controls, mean values were as follows: WBC=4.83±1.04×10³; RBC=5.34±0.34×10⁶; hematocrit=37.97%±2.09; platelets=3.30±0.05×10⁵; prothrombin time=9.68±0.36 sec; partial thromboplastin time=28.78±1.32 sec. In treated animals, mean values were: WBC=6.67±2.26×10³; RBC=5.55±0.26×10⁶; hematocrit=38.18%+1.69; platelets 4.33±0.18×10⁵; prothrombin time=10.88±0.49 sec; partial thromboplastin time=33.06±0.76 sec. Further, blood chemistry analyses also showed no significant differences between treated and control animals, which included: uric acid, triglycerides, cholesterol, creatine kinase, lactate dehydrogenase, SGOT, SGPT, SGGT, alkaline phosphatase, bilirubin, total protein, albumin, phosphate, calcium, and amylase.

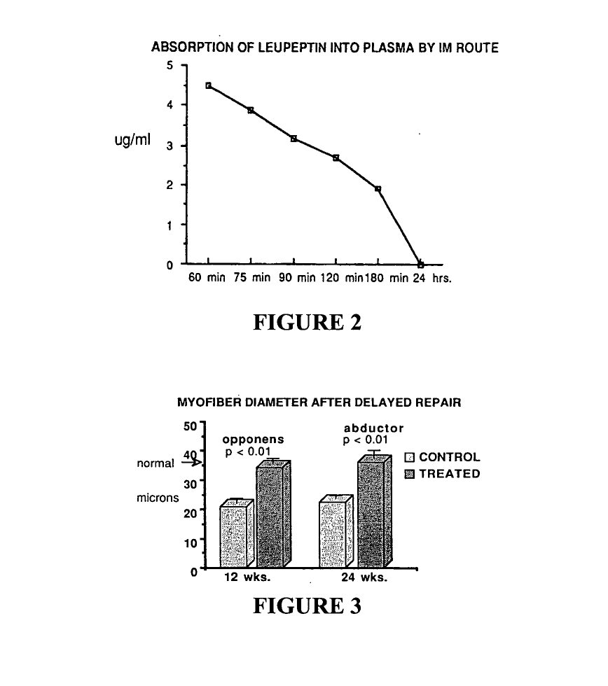

To determine that leupeptin was effectively being absorbed into plasma following intramuscular (“IM”) administration in hind-limbs of 18 mg/kg, blood was withdrawn at 60, 75, 90, 120, 180 minutes and 24 hours after the IM injection (FIG. 2).

Muscle morphology: Fibers biopsied at 24 weeks in the left, unoperated hands of control animals, showed typical histologic structure and a mean diameter of 34.83±1.4 μm and 33.51±2.2 μm, for opponens and abductor myofibers, respectively. Interestingly, opponens and abductor myofibers in the left, unoperated hand of leupeptin treated animals showed normal structure, and were significantly (p<0.01) larger having a mean diameter of 43.25±4.26 μm and 40.43±2.23 μm, respectively.

Opponens pollicis myofibers and abductor pollicis myofibers in the right, nerve-repaired hands of control animals showed histologic and ultrastructural features consistent with denervation atrophy, including small, angulated myofibers and increased fibrosis. However, those muscles in the nerve-repaired hand of treated animals showed histologic and ultra-structural features consistent with control. As shown in FIG. 3, muscles in leupeptin-treated animals showed a statistically significant increase in myofibers diameter, in comparison to control animals.

Opponens and abductor muscles from the left, unoperated hands of all animals showed a mean type II [fast twitch] to type I [slow twitch]) fiber ratio of 2:1. At 12 and 24 weeks after delayed nerve repair, right opponens and abductor muscles in control animals had type II to type I fiber-type ratios of 1.5:1 and 1:1, respectively. Treated animals did not have a similar atrophy of type II myofibers. Treated-animal type II:type I fiber ratios 12 and 24 weeks after delayed nerve repair, were 2.3:1 and 2.5:1 in the opponens and abductor, respectively. Mean differences between treated and control animals were statistically significant, p<0.01.

Muscle biochemistry: Calpain activities were significantly (p<0.01) increased by a mean 50 percent at both 12 and 24 weeks in the right, nerve-repaired opponens and abductor muscles of control animals, when compared to the right opponens and abductor muscles of leupeptin treated animals, indicating direct inhibition of enzyme activity by leupeptin.

Echocardiograms: To assure that leupeptin was causing no pathological effects on heart muscle, the cardiac lateral wall and septum were measured in millimeters in all animals prior to leupeptin treatment and 25 weeks after delayed nerve repair. Prior to treatment, in animals designated as controls, the lateral wall was 2.88±0.33 mm and the septum was 2.84±0.2 mm. Twenty-five weeks later in control animals, the lateral wall was 3.0±0.5 mm and the septum was 2.9±0.2 mm. Prior to treatment, in animals designated to receive leupeptin, the lateral wall was 3.5±0.2 mm and the septum was 2.9±0.4 mm. Twenty-five weeks later, in leupeptin-treated animals, the lateral wall was 3.2±0.1 mm and the septum was 2.8±0.3 mm. Importantly, there were no significant statistical differences within either the control or treatment groups or between the control and treatment groups (data not shown).

The results of this study indicated that leupeptin partially inhibits muscle denervation atrophy after immediate epineural nerve repair in a primate median-nerve model. After a complete median-nerve transection injury in the mid-forearm of primates, with a delay in nerve repair of 3-weeks duration followed by leupeptin treatment, the result was a significant increase in myofiber diameter, with retention of normal structure and less fibrosis. The mechanism of leupeptin action in denervated muscle appears to be the direct inhibition of the calpains, located at the Z-band, sarcolemma, and myofiber basal lamina. After denervation, nerve repair, and leupeptin treatment, the disassembly of the myofiber is prevented by inhibition of the calpains at these sites (data not shown).

### Second Set of Monkey Studies: Oral Administration of Leupeptin

Subsequent to the above studies another series of experiments were performed on the monkeys to assess another route of leupeptin administration in these animals (8). In this regard, oral delivery of leupeptin, after median nerve transection and epineural nerve repair, was studied for its potential benefits to neuromuscular recovery. The results of the controlled, dose-response study indicated that leupeptin was effectively absorbed into plasma by the oral route of administration. When plasma leupeptin concentrations were 3 mg or greater, morphologic and functional motor recovery were facilitated after nerve repair. Serial testing of hematology, blood clotting, and serum biochemistry showed that there were no adverse effects, when leupeptin was administered twice daily for 6 months following nerve repair. These data suggest that leupeptin may be an effective and safe pharmaceutic adjunct to nerve repair, and may have clinical benefits in humans, where the oral route is a much preferred method of delivery.

Hematology/blood chemistry. Blood was drawn from the femoral vein at 1, 3, 4, 5, and 7 months postoperatively. Complete blood counts, clotting parameters, and serum chemistries in control animals and in animals after oral leupeptin administration at 3, 6, 12, and 18 mg/kg were quantified. Values were determined for hemoglobin, hematocrit, WBC, RBC, MCV, MCH, MCHC, differential (polymorphonuclear leukocytes, lymphocytes, monocytes, eosinophils, basophils), platelets, prothrombin time, partial thromboplastin time, glucose, urea nitrogen, creatinine, total protein, albumin, total bilirubin, alkaline phosphatase, SGPT, SGOT, lactic dehydrogenase, cholesterol, calcium, phosphorus, sodium, potassium, chloride, A/G, BUN/creatinine, globulin, amylase. There were no significant differences between controls vs. leupeptin treatment animals. Also, there were no significant differences between leupeptin-treatment groups (data not shown).

Muscle: Opponens pollicis myofibers biopsied at 3 months in the left, unoperated hands of all animals showed typical histologic structure. There were no significant statistical differences between the left control values in comparison to each, left, leupeptin-treatment group.

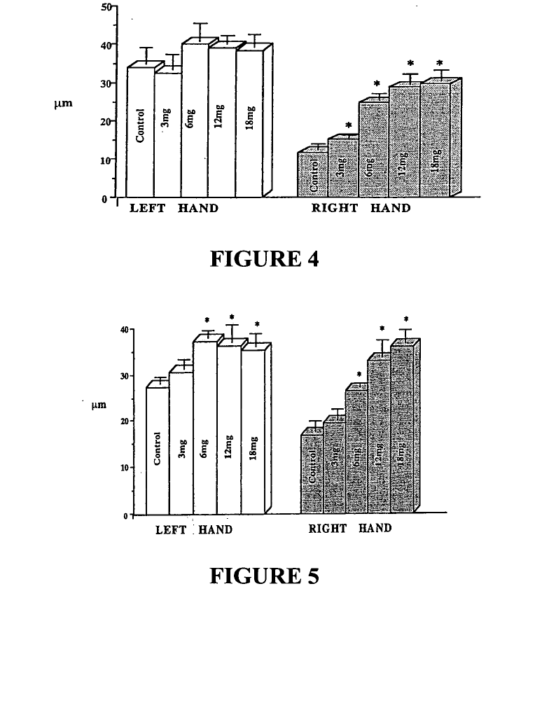

Opponens pollicis myofibers, biopsied from the right hand of all animals at 3 months post nerve repair showed, in the controls, histologic features consistent with denervation atrophy, including small, angulated myofibers and areas of increased fibrosis. Animals in the 3-mg leupeptin treatment group also showed histologic features consistent with denervation atrophy. However, animals in the 6-mg, 12-mg, and 18-mg treatment groups showed histologic features consistent with normal myofibers (data not shown). Myofibers in the 12- and 18-mg/kg groups showed a more typically normal structure than myofibers in the 6-mg group. As shown in FIG. 4, opponens pollicis myofibers in the right hands of leupeptin-treated animals showed increased myofiber diameter, in comparison with the right hands of the control group. The most significant increases occurred at the 6-mg, 12-mg, and 18-mg doses. When expressed as a percentage atrophy, in comparison to the left, unoperated control group, the right control group lost 63 percent of its myofiber diameter; the 3-mg group lost 53% of its myofiber diameter; the 6-mg group 23%; the 12-mg group 11%; and the 18-mg group only 8%.

Histologic examination of the left, unoperated abductor pollicis muscle in all animals revealed typically normal myofibers (FIG. 5). Histologic examination of the right, nerve-repaired hands of controls abductor pollicis muscle biopsied 6 months after nerve repair, still showed evidence of denervation atrophy (FIG. 5).

In comparison to the control group, all leupeptin-treatment abductor pollicis muscle groups demonstrated increased myofiber diameters (FIG. 5). In the 3-mg and 6-mg leupeptin-treatment groups, there were foci of denervated myofibers, more so in the 3-mg than in the 6-mg treatment group (data not shown).

When expressed as a percent atrophy in comparison to the left, unoperated abductor pollicis muscle, the right abductor control group showed a 40 percent loss of myofiber diameter; the 3-mg group, a 30% loss; the 6-mg group, a 5% loss; the 12-mg group, no atrophy but an increase of 1.2% in myofiber diameter; and the 18-mg group, no atrophy but an increase of 1.3% in myofiber diameter. Electron microscopic analysis of muscle samples was reflective of the findings by light microscopic analysis (data not shown).

Muscle calpain activities: Calpain activities in the left and right opponens pollicis and abductor pollicis, 3 and 6 months after right median nerve repair, showed some variability due to the pooling of small sample numbers. However, the trend in activities, as was expected, when comparing control, left muscle to right, denervated, leupeptin-treated muscle, was an overall decrease in calpain activity after leupeptin treatment. The mean decrease in calpain activity in the right opponens pollicis muscles of leupeptin treated animals was 30%. The mean decrease in calpain activity in the right abductor pollicis muscles of leupeptin-treated animals was 24%.

The results of this study indicate that leupeptin, when delivered by the oral route of administration after median nerve transection and microsurgical repair in primates, inhibits muscle denervation atrophy. Calpain inhibition by leupeptin induced morphologic recovery in treated primates, as demonstrated by increased myofiber diameter, with retention of normal myofiber morphology distal to the nerve repair site. The results support the hypothesis that calpain inhibition by leupeptin prevents disassembly of myofibers, with retention of the end-target, skeletal muscle as a neurotrophic target after nerve repair.

An important finding of this study is that leupeptin treatment did not adversely affect hematology, clotting, or serum biochemistry parameters in primates. Finally, the oral method of peptide delivery is much preferred for human application, as any pharmaceutic adjunct to nerve repair will most likely require chronic (6 to 12 months) administration.

### Example 3

### Clinical Studies

Leupeptin's low toxicity, non-immunogenicity and stability profile have made it a potential drug for eventual human use. In this regard, a preliminary safety trial of leupeptin in seven Italian DMD children with ages varying from 7-10 was performed. The trial was approved by the Italian Ministry of Health and the leupeptin supplied by the Nippon Kayaku Co., Japan. As seen in Table 3, leupeptin was administered orally at a total dose of 50 mg/twice daily in six children and 100 mg/twice daily in one child, both doses well below the efficacious dose [15-18 mg/kg/day] established in monkeys. During the process of muscle degeneration, muscle cells break open and their contents find their way into the bloodstream. Because most of the creatine kinase (CK) in the body normally exists in muscle, a rise in the amount of CK in the blood indicates that muscle damage has occurred, or is occurring. CK levels can be slightly elevated (500 U/L) in some nerve disorders, or grossly elevated (5,000 to 15,000 U/L) in DMD or inflammatory myopathies. Importantly the significant decline in CK levels after treatment with leupeptin indicates a significant improvement (lessening of muscle breakdown) in these individuals, especially in the child given 100 mg twice daily.

TABLE 3
TREATMENT OF DUCHENNE PATIENTS WITH LEUPEPTIN

| | | | CREATINE KINASE | |
|---|---|---|---|---|
| | | DOSE (mg) | (Units/100 ml) | |
| PATIENT | AGE (in yrs.) | Twice daily | 0 Time | After 1 yr. |
| R.P | 10 | 50 | 7,055 | 2,500 |
| A.D | 10 | 50 | 11,055 | 3,300 |
| S.M | 9.5 | 50 | 8,520 | 3,500 |
| O.Z. | 7.0 | 50 | 6,360 | 2,500 |
| P.R. | 7.0 | 50 | 9,910 | 4,100 |
| I.M. | 8.0 | 100 | 6,625 | 375 |
| M.S. | 10 | 50 | 6,270 | 2,700 |

Even at this lower dosage after one year of treatment, the measurement of creatine kinase (a routine measure of muscle integrity) showed values that were significantly decreased in 6 children at the 100 mg/day and a very substantial decrease at the 200 mg/day level (twice daily) in one child (these creatine kinase levels approached the upper end of the normal range). Importantly, as was observed in the monkey studies, the drug was well tolerated for one year and no adverse effects seen in any child. Blood chemistries measured over the year showed no abnormalities, and importantly, muscle function tests measured at 6 months and one year showed little decline in muscle strength when compared to the start of the trial.

Despite the study's limitations (subject age, drug dose, lack of power), it

nonetheless demonstrated that: 1) leupeptin appeared to be well tolerated by all of the children participating in the study; and 2) they all had a decrease in creatine kinase even at low doses.

### Example 4

### Studies with Aminocarnityl-Succinyl-Leucyl-Argininal

Aminocarnityl-succinyl-leucyl-argininal, which is Formula IV in the application and referred to below as CLA, is an aminocarnitine formulation of the tripeptide aldehyde leupeptin in which aminocarnitine is used to acylate a portion of the leupeptin molecule following removal of the acetyl-leucyl N-terminal end of the leupeptin molecule by enzymatic cleavage with subsequent covalent linkage of carnitine to the remaining leucyl argininal moiety.

An initial series of toxicology experiments was performed in rats to assess the effect of CLA at various doses, on animal viability. No toxic effect of CLA was observed at any of the doses given. A series of experiments to examine the ability of CLA vs. leupeptin to decrease activated calpain levels and inhibit muscle wasting was conducted. In the experiment shown in Table 4, rats were utilized to assess the effect of IP injection of either leupeptin or CLA into the hind limb muscle on their ability to inhibit calpain activity. To this end, four rats were injected IP with either leupeptin or CLA. After 2 hours, the muscle was isolated, homogenized, and assayed for remaining calpain activity by the standard assay.

TABLE 4

| | % inhibition | % inhibition/μmole | Fold |
|---|---|---|---|
| Control | 0.0 | 0.0 | |
| Leupeptin | 20% | 0.26% | 1 |
| (75 μmoles) | | | |
| CLA | 48% | 3.5% | 13.5 |
| (14 μmoles) | | | |

The results of the experiment shown in Table 4, indicate that CLA is significantly (13.5-fold) more effective at penetrating the cell membrane to inhibit calpain intracellularly per μmole than leupeptin.

In another rat study (data shown in Table 5), the effectiveness of CLA in delaying muscle atrophy as compared to the parent compound leupeptin, was assessed. Denervation was carried out on one of the hind limb sciatic nerves of the rat. Denervation was allowed to proceed for 7 days. Either leupeptin or CLA was injected 2×/day in forelimb muscle for seven days. The non-denervated hind limb was used as the control. Leupeptin and/or CLA were injected into alternating forelimb muscles for a period of seven days after which, animals were sacrificed, muscles excised and weighed.

TABLE 5
In Vivo Comparison of Inhibition of
Muscle Atrophy due to Denervation:
Comparison of Leupertin to CLA.

| | % loss |
|---|---|
| Leupeptin | |
| Control | 32.9 ± 5 |
| 1.5 mg/kg | 30.9 ± 4 |
| 15.0 mg/kg | 2.6 ± 0.3 |
| CLA | |
| Control | 28.0 ± 3 |
| 1.0 mg/kg | 5.2 ± 1 |
| 3.0 mg/kg | <1.0 |
| 6.0 mg/kg | <1.0 |

As shown in Table 5, CLA was able to achieve similar inhibition of muscle wasting at 1 mg/kg as did leupeptin at 15 mg/kg. These results were in agreement with the data presented in Table 4, which showed a 13.5 fold improvement on the in vivo inhibition of calpain by CLA. These studies demonstrate that CLA can effectively target the attached leupeptin fragment to the muscle so as to allow for a very effective inhibition of calpain activity, and amelioration of muscle atrophy due to denervation. All rats remained healthy without any apparent negative toxic effect of either CLA or leupeptin.

CLA was injected parenterally daily at the indicated concentrations into mdx mice from one to two months. Mdx control mice were injected with saline alone. At two months, the animals were sacrificed and indicated muscles removed, fixed, and stained for myofiber diameter analysis. It can be seen that CLA (formula IV) at both concentrations in both the diaphragm (Table 6) and gastrocnemins muscle (Table 7) significantly improved the retention of muscle size even at 5 mg/kg. This is in contrast to leupeptin alone, which showed efficacy only at 12-18 mg/kg twice daily. Thus, CLA is at least five to seven times more effective than leupeptin alone.

TABLE 6
(Diaphragm)

| | Amount of | | |
|---|---|---|---|
| Mouse type | compound III | | Myofiber diameter (mm) |
| normal (control) | 0 | | 33.5 ± 5.6 |
| mdx | 0 | | 18.6 ± 6.1 |
| Mdx | 5 | mg | 25.1 ± 7.3 |
| Mdx | 10 | mg | 25.4 ± 7.3 |

TABLE 7
(Gastrocnemis)

| | Amount of | | |
|---|---|---|---|
| Mouse type | compound III | | Myofiber diameter (mm) |
| normal (control) | 0 | | 34.3 ± 6 |
| mdx | 0 | | 18.7 ± 6 |
| mdx | 5 | mg | 28.7 ± 7 |
| mdx | 10 | mg | 30.6 ± 11 |

### Example 5

### Conversion of Aminocarnityl-Succinyl-Leucyl-Argininal Diethyl Acetal (Cla Acetal) to Aminocarnityl-Succinyl-Leucyl-Argininal (Cla) Using Acid as an Activating Component

To illustrate how an acid component could be used as part of a kit to generate the active aldehyde compound, experiments were performed to measure conversion of the diethyl acetal to aldehyde by incubating the acetal in solutions of variable acidity for variable lengths of time.

Solutions of CLA diethyl acetal in water were prepared at a concentration of 5 mg/mL and the pH was adjusted to values shown in Table 8 using concentrated hydrochloric acid. Each solution was incubated at different times at room temperature. Samples were removed at each time point and quenched using basic phosphate buffer, yielding a final pH of 9. The quenched samples were analyzed using an Agilent 1100 HPLC system. The aldehyde was chromatographically separated from the acetal on a C18 reverse-phase HPLC column, using a linear gradient where the starting mobile phase was 0.1% trifluoroacetic acid in a 99:1 water:acetonitrile mixture and the final mobile phase was 0.1% trifluoroacetic acid in a 50:50 water:acetonitrile mixture. The absorbance at 220 nm was monitored, and the area of the peak corresponding to elution of the CLA diethyl acetal was computed. The results are shown in Table 8. These results show that CLA acetal can be quantitatively and rapidly converted to an active aldehyde form using acid.

TABLE 8
Conversion of CLA acetal to aldehyde at pH 0 and pH 1

| | | Activation | Percent | Approximate |
|---|---|---|---|---|
| | Relative Peak | Time | acetal | t_{1/2} for |
| pH Condition | Area | (min) | remaining | activation |
| pH = 0 | 225 | 0 | 100.0 | 10 minutes |
| | 180 | 5 | 80.0 | |
| | 64.2 | 15 | 28.5 | |
| | 25.3 | 30 | 11.2 | |
| | 25.4 | 45 | 11.3 | |
| | 25.5 | 60 | 11.3 | |
| | 20.2 | 90 | 9.0 | |
| | 24.5 | 120 | 10.9 | |
| | 20.9 | 240 | 9.3 | |
| | 20.5 | 1440 | 9.1 | |
| pH = 1.0 | 315 | 0 | 100.0 | 2.5 hours |
| | 317 | 5 | 100.6 | |
| | 306.6 | 15 | 97.3 | |
| | 290.4 | 30 | 92.2 | |
| | 273.7 | 45 | 86.9 | |
| | 257.4 | 60 | 81.7 | |
| | 229.1 | 90 | 72.7 | |
| | 204 | 120 | 64.8 | |
| | 132.9 | 240 | 42.2 | |
| | 20.2 | 1440 | 6.4 | |

### Example 6

### Qualitative Measurement of the Oral Bioavailability of the CLA Acetal in the Rat

This experiment was designed to measure in a qualitative manner the oral bioavailability of CLA acetal in rats. Four Lewis strain normal male rats, 9 weeks of age were administered a single dose of 50 mg/kg of the acetate salt of the CLA acetal compound, using a single oral gavage. Blood draws were taken at either 45 minutes or 1 hour after administration of drug. The blood samples were immediately centrifuged at 3000 rpm for 3-5 minutes to separate plasma from cells and the plasma and cells were frozen separately by immersion in liquid nitrogen and storage in a −20° C. freezer.

Selectively deuterated forms of the CLA acetal and aldehyde were added to each plasma sample at a final concentration of 150 ng/mL prior to extraction, to serve as internal standards in the analysis of drug levels. The aldehyde and acetal forms of CLA were then extracted from the plasma using a Bligh Dyer-type liquid phase extraction followed by solid-phase extraction using a reverse phase column. The relative amounts of CLA acetal and aldehyde were then measured using HPLC separation with mass spectrometric detection, using an electrospray ionization source and a Micromass Qtof detector. The peak areas were integrated and the quantity of compound found in plasma was estimated by comparison to the deuterated internal standards. Although this experiment is qualitative and does not yield a pharmacokinetic profile for the drug, the high levels of acetal found at a single time point in the blood of all of the rats indicates that the CLA acetal is absorbed intact when administered orally. The relatively high levels observed also indicate that, if conversion of prodrug to drug occurs after the CLA acetal enters the bloodstream, its oral bioavailability is likely to be high enough to provide a therapeutic benefit to human patients.

TABLE 9
CLA acetal and aldehyde concentrations of
acetal and aldehyde in rat plasma.

| | | | Estimated Plasma |
|---|---|---|---|
| | | Estimated Plasma | Concentration of |
| | | Concentration of CLA | CLA aldehyde |
| Rat | Time (minutes) | acetal (ng/mL) | (ng/mL) |
| 1 | 45 | 5,840 | <20 |
| 2 | 60 | 970 | <20 |
| 3 | 60 | 8240 | 72 |
| 4 | 60 | 51200 | <20 |

### Example 7

### Study of Toxicity of CLA Acetal when Administered Orally to Rats as a Single Dose

Sprague Dawley rats were administered single doses of CLA acetal by oral gavage at 7-8 weeks of age. The rats were divided into 4 groups, consisting of equal numbers of male and female rats, as shown in Table 10. Each group received a single oral dose level of CLA acetal, ranging in amount from 0 to 2000 mg/kg. The vehicle consisted of Dulbecco's phosphate-buffered saline. No evidence of toxicity was observed at any of the dose levels at any time up to 24 hours after dosing.

TABLE 10
Single-dose oral rat toxicology study.

| | | | | | Number of | |
|---|---|---|---|---|---|---|
| | | | Concen- | Dose | Animals | |
| Group | | Dose Level | tration | Volume | | Fe- |
| Number | Treatment | (mg/kg/day) | (mg/mL) | (mL/kg) | Males | males |
| 1 | Vehicle | 0 | 0 | 10 | 5 | 5 |
| 2 | Low Dose | 250 | 25 | 10 | 5 | 5 |
| 3 | Mid Dose | 1000 | 100 | 10 | 5 | 5 |
| 4 | High Dose | 2000 | 200 | 10 | 5 | 5 |

### Example 8

### Study of Toxicity of CLA when Administered Intravenously to Rats as a Single Dose

In a pilot toxicology study, two groups of 2 male and 2 female Sprague-dawley rats each were administered intravenous bolus doses of 250 and 500 mg/kg CLA. CLA aldehyde was found to be lethal at the 500 mg/kg dose level, with death occurring in 1 to 5 minutes. In a larger, follow-up study (Table 11), Sprague-Dawley rats were administered several lower doses of CLA intravenously at 7-8 weeks of age. The rats were divided into 4 groups consisting of equal numbers of male and female rats. Each group received a different dose level of CLA, as shown in Table 11, ranging from 0 to 200 mg/kg. The vehicle consisted of Dulbecco's phosphate-buffered saline. The no adverse effect limit (NOAEL) was found to be 100 mg/kg in this study, and the maximum tolerated dose was 200 mg/kg. No toxicity was observed at 50 and 100 mg/kg.

TABLE 11
Single-dose intravenous toxicology study

| | | | Concen- | | | |
|---|---|---|---|---|---|---|
| | | | tration | Dose | Number of | |
| Group | | Dose Level | (mg/ | Volume | Animals | |
| Number | Treatment | (mg/kg/day) | mL) | (mL/kg) | Males | Females |
| 1 | Vehicle | 0 | 0 | 10 | 5 | 5 |
| 2 | Low Dose | 50 | 25 | 10 | 5 | 5 |
| 3 | Mid Dose | 100 | 50 | 10 | 5 | 5 |
| 4 | High Dose | 200 | 100 | 10 | 5 | 5 |

### Example 9

### Measurement of the Inhibition of Calpain Activity by CLA in Human Muscle Cells

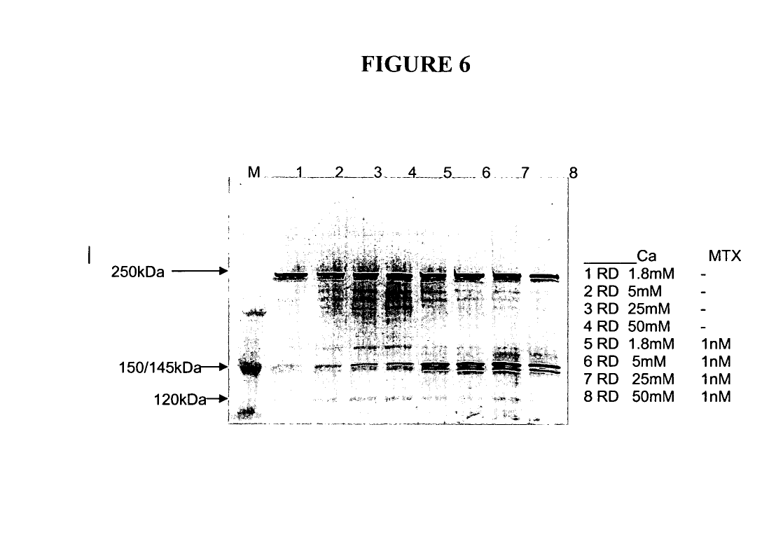

This study was designed to measure the effect of CLA, a calpain inhibitor, upon calpain activity inside living muscle cells. Alpha-II spectrin, a large cytoskeletal protein, is known to be a substrate of calpain, which is activated following calcium influx triggered by cellular damage or necrosis. Calcium-dependent cell death also occurs in many debilitating muscle diseases. Full length alpha-II spectrin has a molecular weight of approximately 250 kilodaltons; upon cleavage by calpain, calpain-specific spectrin breakdown products with molecular weights of approximately 150 and 145 kilodaltons, respectively, are generated sequentially. Human RD muscle cells are a rhabdomyosarcoma muscle cell line that expresses OCTN2, a transporter for carnitine. By monitoring the reduction in spectrin breakdown products caused by calpain inhibition in RD muscle cells, it is possible to measure the potency of inhibitors according to the extent to which they reduce the appearance of calpain-specific spectrin breakdown products. FIG. 6 shows the appearance of spectrin breakdown products in RD cells that have been incubated with maitotoxin in the presence of calcium.

In the experiment, Rd muscle cells were incubated with either CLA or leupeptin for 1 hour. 1 nM maitotoxin was added to the cells to stimulate calcium influx and calpain activation. The cells were incubated for another 60 minutes. The cells were then lysed in phosphate buffer containing 1% Triton X-100 and a protease inhibitor cocktail. The lysed samples were centrifuged and the supernatants were collected. The protein concentration in each supernatant was measured using a standard assay. 20 micrograms of each sample was electrophoresed on a 4% Tris-glycine polyacrylamide gel. Western blotting was performed using an antibody to alpha II spectrin that detects the full-length spectrin as well as the 150 and 145 kilodalton fragments.

Table 12 shows the results of the experiment comparing leupeptin (a peptide aldehyde with the sequence leu-leu-argininal) to CLA (aminocarnitine-leu-argininal). The IC₅₀ of CLA is shown to be approximately 200-fold lower than that of leupeptin. This result is attributable to improved transport of CLA into muscle cells due to the aminocarnitine moiety.

TABLE 12
IC₅₀ values for inhibition of spectrin breakdown product formation.

| | IC₅₀, 150 kDa | IC₅₀, 145 kDa |
|---|---|---|
| Compound | fragment (μM) | fragment (μM) |
| Leupeptin | 80,800 | 5750 |
| CLA aldehyde | 356 | 37 |
| Ratio of IC50s (leupeptin:CLA) | 227:1 | 155:1 |

### Example 10

### Uptake and Conversion of CLA in Rat Muscle

This experiment was designed to measure the uptake of CLA acetal in rat gastrocnemius muscle and to detect conversion of CLA acetal to CLA as it occurs in rat muscle. Four Lewis strain normal male rats, 9 weeks of age were administered a single dose of 50 mg/kg of the acetate salt of the CLA acetal compound, using a single oral gavage. At different times following administration of drug, each rat was sacrificed, the left and right gastrocnemius muscles were dissected from the rat, and the muscles were frozen by immersion in liquid nitrogen and storage in a −80° C. freezer.

The muscle tissues were thawed and homogenized using a blender. Selectively deuterated forms of the CLA acetal and CLA were added to each muscle homogenate at a final concentration of 150 ng/mL prior to extraction, to serve as internal standards in the analysis of drug levels. The aldehyde and acetal forms of CLA were then extracted from the muscle homogenates using a Bligh Dyer-type liquid phase extraction followed by solid-phase extraction using a reverse phase column. The relative amounts of CLA acetal and CLA were then measured using HPLC separation with mass spectrometric detection, using an electrospray ionization source and micromass Qtof detector. The peak areas were integrated and the quantity of compound found in the homogenates was estimated by comparison to the deuterated internal standards.

The concentrations of CLA acetal and CLA shown in Table 13 indicate that most of the CLA taken up by muscle tissue is detected as the active aldehyde drug within 2 hours of oral administration of the inactive CLA acetal. These data show that CLA acetal is likely to be a prodrug that targets muscle tissue, where it is activated, reducing the risk of potential toxicity due to exposure of other tissues to the active drug form.

TABLE 13

| | | | Muscle | Muscle | |
|---|---|---|---|---|---|
| | | | concentration | concentration | Percent of |
| | | | of prodrug, ng | of active drug, | drug present |
| Rat | Muscle | Time | per gram | ng per gram | as the active form |
| 1.1 | Left gastrocnemius | 2 hours | 37.4 | 124.6 | 76.9 |
| 1.3 | Right gastrocnemius | 6 hours | 9.7 | 683.6 | 98.6 |
| 2.1 | Right gastrocnemius | 8 hours | 9.2 | 330.8 | 97.3 |
| 2.3 | Right gastrocnemius | 8 hours | 10.3 | 758.4 | 98.7 |

### Example 11

### Additional Data for Measurement of the Inhibition of Calpain Activity by CLA in Human Muscle Cells

The study previously described in Example 9, i.e. the measurement of the effects of CLA on inhibition of calpain breakdown of alpha II spectrin in rhabdomyosarcoma mucle cell cultures, was repeated to collect additional data, to obtain a total of five independent measurements of each value. The experimental methods were identical to those described in Example 9. The total averaged results, including those from Table 12, are shown in table 13. These averaged results, despite the relatively large calculated standard errors, nevertheless show that the “carnitine-targeted” peptide-aldehyde CLA (in its parent aldehyde form) is a very significantly more active calpain inhibitor than the previously known peptide aldehyde Leupeptin.

TABLE 13
IC₅₀ values for inhibition of spectrin breakdown product formation.

| | | | IC₅₀, | |
|---|---|---|---|---|
| | IC₅₀, 150 kDa | Standard | 145 kDa | Standard |
| | fragment | Error | fragment | Error |
| Compound | (μM) | (μM) | (μM) | (μM) |
| Leupeptin | 51,900 | 20,000 | 3,800 | 1500 |
| CLA aldehyde | 397 | 113 | 70 | 38 |
| Ratio of IC50s | 131:1 | | 54:1 | |
| (leupeptin:CLA) | | | | |

### Example 12

### Measurement of the Inhibition of Calpain Activity, using an Enzymatic Assay, for the L-Argininal and D-Argininal isomers of CLA

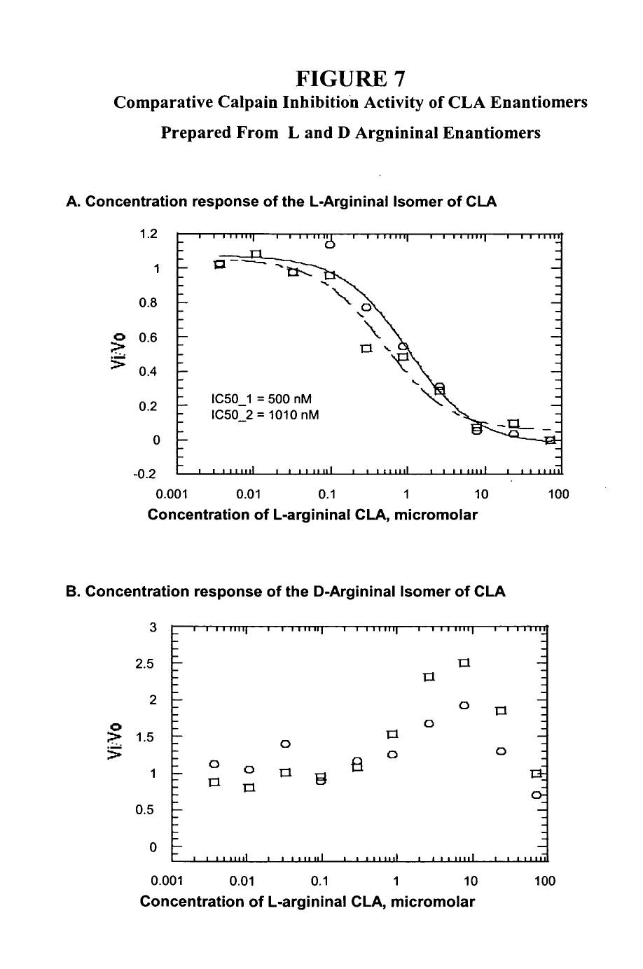

The α-carbon stereocenter adjacent to the aldehyde group of CLA can be prepared in a “natural” L or “unnatural” D configuration. It is known however that enolization of such aldehydes can easily occur during synthesis, storage, or administration of such peptide aldehydes to patients, with the result that the α-carbon stereocenter undergoes racemization. To investigate the relative biological activity of the optical isomers of CLA, the pure form of both the L-argininal diethyl acetal and D-argininal diethyl acetal forms of CLA were prepared via the method described in Example 11 below using both L and D enantiomers of the argininal acetal starting materials. Each acetal stereoisomer was then hydrolyzed to its corresponding free aldehyde by dissolving the compounds in 0.5 N hydrochloric acid to a final concentration of 5 mM, and incubating the solution for 1 hour at 22° C. The reactions were stopped by raising the pH to 2 by dropwise addition of sodium hydroxide. The resulting aldehyde solutions were then dissolved in 50 mM HEPES buffer, pH 7.4, and varying concentrations were incubated for 20 minutes with porcine kidney calpain 2 (EMD Biosciences, San Diego Calif., catalog #208715) at 37° C. Calcium chloride was added to a final concentration of 10 mM, and the samples were incubated for additional 4 minutes, in a 96-well black polystyrene plate. A calpain substrate, DABCYL-Thr-Pro-Leu-Lys-Ser-Pro-Pro-Pro-Ser-Pro-Arg-EDANS (EMD Biosciences, catalog #208771, DABCYL=4-((4-(dimethylamino)phenyl)azo)benzoic acid, EDANS=5-((2-aminoethyl)amino)naphthalene-1-sulfonic acid) was added to a final concentration of 30 micromolar. The plate was transferred to a Victor-III plate reader (Perkin-Elmer), and the increase in fluorescence due to hydrolysis of the substrate was monitored using an excitation bandpass filter centered at 360 nm and an emission bandpass filter centered at 460 nm. Slopes were calculated for the initial phase of each reaction and the ratios of velocities for each compound concentration (Vi) to the uninihibited reaction velocity (V₀) were calculated, plotted, and fitted to a rectangular hyperbola using the program Kaleidagraph (Synergy software). The plotted data for titrations of Calpain-2 with each isomer of CLA are shown in FIG. 7. Panel A of FIG. 7 shows the fitted data for two titrations of the L-isomer, whereas Panel B shows a plot of data for two titrations of the D-argininal isomer. When a compound is inhibitory to the enzyme, it is expected to cause the ratio of compound velocity to uninihibited reaction velocity (ViNo) to decrease, and the concentration required to inhibit 50% of the enzyme activity (IC₅₀) can then be calculated as the concentration where Vi/Vo=0.5. Whereas the L-isomer of CLA caused a hyperbolic decrease in Vi/Vo that is consistent with competitive inhibition of calpain, with an IC₅₀ in the 500-1000 nM range (Panel A.), the D-isomer caused no such decrease and appears to be a poor inhibitor of Calpain-2, with an IC₅₀ that is likely to be greater than 100 micromolar (Panel B.). An increase in enzyme activity appears to occur at intermediate concentrations of the D-isomer (Panel B.). This increase may be due to weak binding at an allosteric site that is not inhibitory the enzyme.

### Example 13

### Synthesis of CLA Acetal (Aminocarnityl-Succinyl-Leucyl-Argininal Diethyl Acetal, Monoacetate)

CLA acetal (aminocarnityl-succinyl-leucyl-argininal diethyl acetal, as the monoacetate salt) can be synthesized by the following seven step synthesis.

### Step 1: Synthesis of L-Aminocarnitine4 ethyl ester (EtO-L-Aminocarnitine) by Esterification

The R and S isomers of aminocarnitine are available from Sigma-Tau Industrie Farmaceutiche Riunite S.p.A. of Rome, Italy, or can be prepared by the methods disclosed by U.S. Pat. No. 6,822,115, which is hereby incorporated herein by this reference for its teachings of methods of the synthesis of aminocarnitine derivatives and its precursors. L-Aminocarnitine chloride hydrochloride salt is esterified with ethanol (absolute) at a temperature between −15° C. and +20 C using hydrogen chloride gas. The solvent is removed under reduced pressure, and the resulting product is used directly in the next step.

(Yield approx. 100%)

### Step 2: Coupling of EtO-L-Aminocarnitine and Succinyl-Leucyl Benzyl Ester by a Mixed Anhydride Method. Synthesis of EtO-L-Aminocarnityl-Suc-Leu-OBzl

The benzyl ester of leucyl succinnate (Suc-Leu-OBzl) can be prepared by condensing the benzyl ester of L-Leucine with succinic anhydride. The first step product (EtO-L-Aminocarnitine) is coupled with Suc-Leu-OBzl in dimethylformamide (DMF) at a temperature between −25° C. and +20 C) using pivaloyl chloride as coupling reagent and N-methylmorpholine and pyridine as bases. The reaction mixture is concentrated under reduced pressure to remove DMF. The resulting oil is dissolved in water/ethyl acetate whereupon phase separation occurs. The aqueous layer is washed with ethyl acetate and then extracted with n-butanol. The n-butanol layers are washed with water and combined. The solvent is removed under reduced pressure, and the resulting product (EtO-L-Aminocarnityl-Suc-Leu-OBzl) is used directly for the next step.

(Yield approx. 80%)

### Step 3: Synthesis of EtO-L-Aminocarnityl-Suc-Leu-OH by Catalytic Hydrogenation

The benzyl protecting group of EtO-L-Aminocarnityl-Suc-Leu-OBzl is removed by catalytic hydrogenation using supported palladium as catalyst in DMF at ambient temperature. On completion of the reaction, the catalyst is filtered off and the mixture is used directly in the next coupling step.

(stage 5)

(Yield approx. 100%)

### Step 4: Synthesis of H-Arg(Z)₂-aldehyde diethyl acetal

Fmoc-Arg((Z)₂-aldehyde diethyl acetal (available from Bachem A G, Bubendorf Switzerland, Fmoc=9-Fluorenylmethyloxycarbonyl protecting group, Z=benzyloxycarbonyl protecting group) is deprotected with piperidine in DMF at ambient temperature to selectively remove the Fmoc protecting group. On completion of the reaction, the reaction mixture is precipitated by addition of an ice/water mixture. The precipitate is filtered off and washed with DMF/water. Mother liquor and washing liquors are combined and extracted with IPE. The organic layers are combined and washed with water. The solvent is removed under pressure, and the resulting product is used directly in the next coupling step (stage 5).

(Yield approx. 50%)

### Step 5: Synthesis of EtO-L-Aminocarnityl-Suc-Leu-Arg(Z)-2-aldehyde diethyl acetal by Mixed Anhydride Coupling

The step 3 product (EtO-L-Aminocamityl-Suc-Leu-OH) is coupled with the step 4 product (H-Arg(Z)₂-aldehyde diethyl acetal) in DMF at a temperature between −25° C. and +20° C. using pivaloyl chloride as coupling reagent and N-methylmorpholine and pyridine as bases. The reaction mixture is diluted with water and methyl tertiary butyl ethere (MTBE) whereupon phase separation occurs. The aqueous layer is washed with MTBE, then the pH is adjusted to 8 with sodium hydroxide solution. The aqueous layer is washed again with isopropyl acetate and isopropyl acetate/ethyl acetate. To the aqueous layer, saturated sodium chloride solution is added. It is then acidified to pH 4.5 with hydrochloric acid and extracted with ethyl acetate. After dilution with MTBE, the organic layer is washed with diluted sodium chloride solution. The solvent is removed under reduced pressure, and the resulting product is used directly in the next step.

(Yield approx. 75%)

### Step 6: Synthesis of L-Aminocarnityl-Suc-Leu-Arg(Z)-aldehyde diethyl acetal by Saponification

The ethyl protecting group of the step 5 product (EtO-L-Aminocamityl-Suc-Leu-Arg(Z)-aldehyde diethyl acetal) is selectively removed by saponification with sodium hydroxide solution in ethanol at ambient temperature. On completion of the reaction, the reaction mixture is diluted with water/IPE whereupon phase separation occurs. The aqueous layer is washed with IPE and ethyl acetate. After saturation with sodium chloride, the pH of the aqueous layer is adjusted to 4.5 with hydrochloric acid. The aqueous layer is then extracted with sec-butanol. The solvent is removed under pressure. The crude product is dissolved in purified water and transferred into the acetate salt by ion exchange. The product-containing fractions are combined and directly used in the final stage.

(Yield approx. 100%)

### Step 7: Synthesis of L-Aminocarnityl-Suc-Leu-Arg-aldehyde diethyl acetal, acetate salt (CLA Acetal)

The benzyloxycarbonyl protecting group of the step 6 product (L-Aminocarnityl-Suc-Leu-Arg(Z)-aldehyde diethyl acetal) is removed by hydrogenation using a supported palladium catalyst in purified water/acetic acid at ambient temperature. On completion of the reaction, the catalyst is filtered off and the filtrate is washed with di-isopropyl ether (IPE) to remove the toluene formed by the deprotection reaction. The solvent is then removed under reduced pressure.

The crude CLA acetal is purified by countercurrent distribution (CCD) with an n-butanol/purified water/acetic acid system. The solvent of the pooled fractions is removed under reduced pressure. The resulting oil is co-evaporated with isopropanol (IPA), dissolved in IPA, and precipitated by addition to diethyl ether. The precipitate is filtered off, washed with diethyl ether, and dried under reduced pressure.

(Yield approx. 60%)

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

What is claimed:

1. A kit for treating or preventing a muscle disorder comprising:
a) an acetal prodrug of a compound, or a pharmaceutically acceptable salt or ester thereof, in an amount effective to treat or prevent a muscle disorder in a subject, wherein the acetal prodrug comprises a carrier molecule and one or more protease inhibitors comprising an acetal of an aldehyde group, and
b) an activator for converting the acetal prodrug of the compound to the aldehyde form of the compound;
wherein the carrier molecule has the structure 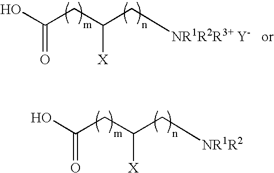
wherein each R¹, R², and R³ is independently selected from the group consisting of hydrogen or a branched- or straight chain alkyl group having 1 to 6 carbon atoms,
X is an O or NR⁶ group, wherein R⁶ is hydrogen or a branched- or straight-chain alkyl group having 1 to 6 carbon atoms;
m and n are independently selected from an integer from 1 to 10, and
Y is a pharmaceutically-acceptable anion; and
wherein X is directly bonded to the one or more protease inhibitors or X is indirectly bonded to the one or more protease inhibitors through a linker comprising up to 25 carbon atoms.

2. The kit of claim 1, wherein m and n are independently selected from the integers 1 to 3.

3. The kit of claim 1, wherein m and n are 1.

4. The kit of claim 3, wherein X is an oxygen atom, directly bonded to the one or more protease inhibitors.

5. The kit of claim 3, wherein X is an oxygen atom, indirectly bonded to the one or more protease inhibitors through the linker.

6. The kit of claim 1, wherein the carrier molecule, or a pharmaceutically acceptable salt thereof, has the structure 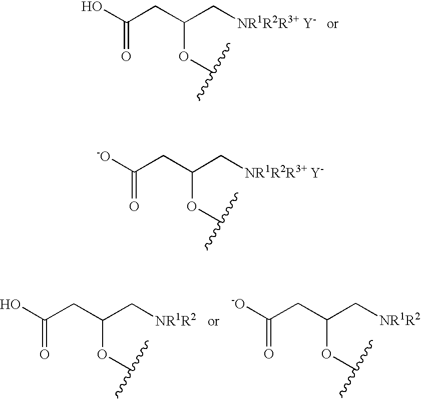
wherein R¹, R², and/or R³ are independently selected from the group consisting of hydrogen or methyl groups, Y⁻ is a pharmaceutically acceptable anion, and the indicted oxygen atom is directly or indirectly bonded to the protease inhibitor.

7. The kit of claim 3, wherein X is an NH group, directly bonded to the one or more protease inhibitors.

8. The kit of claim 3, wherein X is an NH group, indirectly bonded to the one or more protease inhibitors through the linker.

9. The kit of claim 1, wherein the carrier molecule or a pharmaceutically acceptable salt thereof has the structure 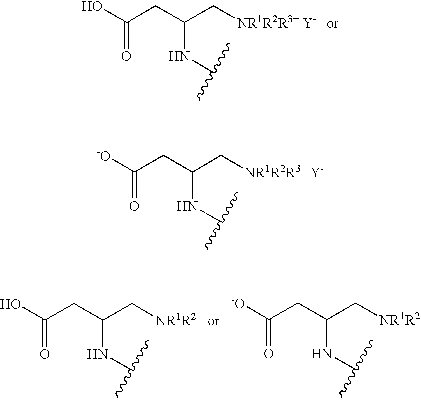
wherein R¹, R², and/or R³ are independently selected from the group consisting of hydrogen or C₁-C₃ alkyl groups, Y⁻ is a pharmaceutically acceptable anion, and the NH group is directly or indirectly bonded to the protease inhibitor.

10. The kit of claims 1, 3, and 9, wherein R¹, R², and R³ are methyl.

11. The kit of claim 1 wherein the protease inhibitor comprises an acetal prodrug of a peptide aldehyde.

12. The kit of claims 1 and 9, wherein the protease inhibitor has the structure 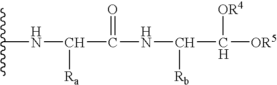
wherein R⁴ and R⁵ are a branched- or straight chain alkyl group having one to three carbon atoms, or wherein R⁴ and R⁵ together form an alkylene residue that forms a five or six membered ring, and wherein Rₐ is hydrogen or a C₁-C₄ alkyl, and R_{b} is hydrogen, a C₁-C₄ straight or branched alkyl, or has one of the structures 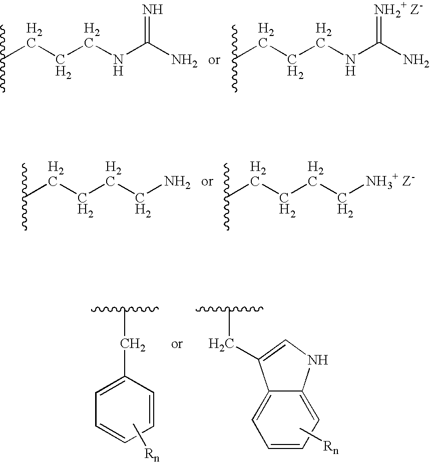
wherein Rₙ is hydrogen, hydroxyl, methyl, ethyl, methoxy, —NH₂, —NHCH₃, —N(CH₃)₂, —SH, or —SCH₃, and wherein Z- is a pharmaceutically acceptable anion.

13. The kit of claim 12, wherein Rₐ is an isopropyl, isobutyl, or 2-butyl group.

14. The kit of claim 12, wherein R_{b} is an isopropyl, isobutyl, or 2-butyl group.

15. The kit of claim 12, wherein R_{b} has the structure

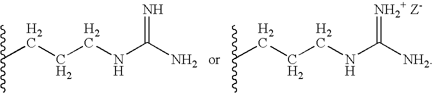

16. The kit of claim 12, wherein R_{b} has the structure

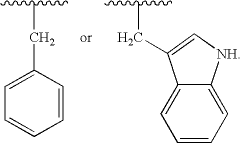

17. The kit of claim 12, wherein R⁴ and R⁵ are ethyl.

18. The kit of claim 1, wherein the protease inhibitor comprises an acetal prodrug of a calpain inhibitor.

19. The kit of claim 1 wherein the prodrug of the calpain inhibitor is an acetal of leucyl-argininal.

20. The kit of claims 1 and 9, wherein the protease inhibitor has the structure 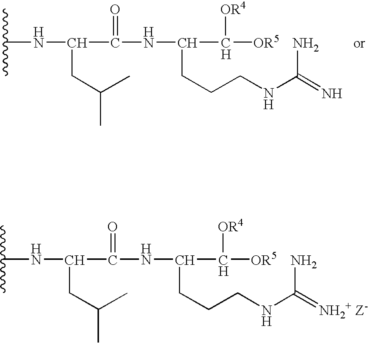
wherein R⁴ and R⁵ are a branched- or straight chain alkyl group having one to three carbon atoms, or wherein R⁴ and R⁵ together form an alkylene residue that forms a five or six membered ring, and wherein Z- is a pharmaceutically acceptable anion.

21. The kit claim 20, wherein R⁴ and R⁵ are ethyl.

22. The kit of claim 1 and 9, wherein the linker comprises a polyalkylene group, a polyether group, a polyamide group, a polyimino group, a polyester, an aryl group, or a polythioether group.

23. The kit of claims 1 and 9, wherein the linker has the formula —C¹(O)(CH₂)ₒ(O)C²—, wherein o is an integer of from 1 to 10, wherein C¹ is covalently bonded to the carrier molecule and C² is covalently bonded to the protease inhibitor.

24. The kit in claim 23, wherein o is from 1 to 5.

25. The kit in claim 23, wherein o is 2.

26. The kit of claim 1, wherein the prodrug has the formula III 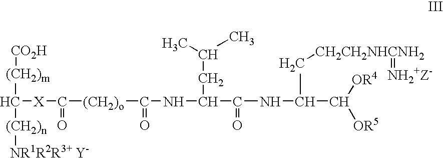
wherein each R¹-R³ is, independently, hydrogen or a branched- or straight chain alkyl group,
R⁴ and R⁵ are, independently, hydrogen or a branched- or straight chain alkyl group, or
R⁴ and R⁵ can be part of a ring,
X is O or NR⁶, wherein R⁶ comprises hydrogen or a branched- or straight-chain alkyl group;
Y and Z are a pharmaceutically-acceptable anion; and
m, n, and o can be an integer from 1 to 10,
or a pharmaceutically-acceptable salt or ester thereof.

27. The kit of claim 26, wherein m and n are 1, and o is 1, 2, or 3.

28. The kit of claim 26, wherein m and n are 1, R¹-R³ are methyl, and X is NH.

29. The kit of claim 26, wherein the compound has the formula III, wherein m and n are 1, R¹-R³ are methyl, R⁴ and R⁵ are ethyl, X is NH, and o is 2.

30. The kit of claim 1, wherein when the protease inhibitor is leucyl-argininal wherein both the leucyl and argininal residues are present in the form of the substantially pure L-enantiomer.

31. The kit of claim 1, wherein the carrier molecule is present in the form of the substantially pure L-enantiomer.

32. The kit of claim 1, wherein the activator comprises one or more of an acid, an enzyme, a metal, a salt, a polymer, a detergent, a zeolite, or a mixture thereof.

33. The kit of claim 1, wherein the activator comprises an acid.

34. The kit of claim 1, wherein the activator comprises hydrochloric acid, phosphoric acid, or a mixture thereof.

35. The kit of claim 1, wherein the prodrug is present in the form of a solution.

36. The kit of claim 35, wherein the solution is an aqueous solution.

37. The kit of claim 1, wherein the acetal prodrug is present as a solid pharmaceutical composition also comprising a pharmaceutically acceptable carrier.

38. The kit of claim 1, wherein the activator comprises HCl or phosphoric acid and the acetal prodrug has the structure 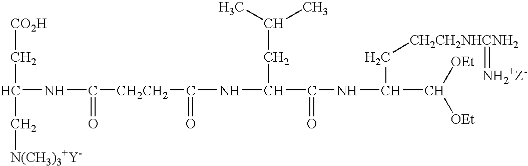
wherein Y and Z are pharmaceutically-acceptable anions.

39. The kit of claim 1, wherein the kit further comprises a neutralizing agent.

40. The kit of claim 39, wherein the neutralizing agent comprises a base.

41. The kit of claim 1, wherein the kit further comprises a heating device.

42. A method for using the kit of claims 1 and 38, for treating or preventing a muscle disorder in a subject, comprising (a) admixing the acetal prodrug of the compound and the activator, to produce at least some of the aldehyde form of the compound and (b) administering to the subject the aldehyde form of the compound in an amount effective to treat or prevent the muscle disorder.

43. The method of claim 42, wherein at least about 90% of the acetal prodrug is converted to the aldehyde form of the compound prior to administration to the subject.

44. The method of claim 42, wherein the aldehyde form of the compound is administered to the subject intravenously, or parenterally.

45. The method of claim 42, wherein the aldehyde form of the compound is administered to the subject orally.

46. The method of claim 42, wherein the muscle disorder comprises a muscular dystrophy, muscle wasting disease, cancer cachexia, cardiomyopathy, cardiac ischemia, denervation atrophy, or AIDS-related muscle wasting.

47. The method of claim 42, wherein the muscle disorder is myotonic, or limb-girdle muscular dystrophy.

48. The method of claim 42, wherein the muscle disorder is Duchenne muscular dystrophy.

49. The method of claim 42, wherein the subject is a mammal.

50. The method of claim 42, wherein the subject is a human male diagnosed as having Duchenne muscular dystrophy.

51. The method of claim 42, wherein the admixing is carried out to form the aldehyde form of the compound and then the aldehyde form of the compound is subsequently further formulated prior to administration, or the admixing is carried out by the subject immediately preceding self administration.

52. The method of claim 42, wherein the aldehyde form of the compound is administered to the subject in an amount of at least about 0.1 mg/kg/day.

53. A pharmaceutical composition for treating or preventing a muscle disorder comprising:
a) an acetal prodrug compound or a pharmaceutically acceptable salt or ester thereof, in an amount effective to treat or prevent a muscle disorder in a subject, wherein the compound comprises a carrier molecule and one or more protease inhibitors comprising an acetal of an aldehyde group, and
b) one or more pharmaceutically acceptable carriers;
wherein the carrier molecule has the structure 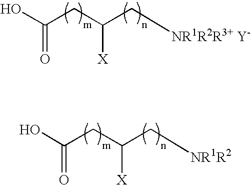
wherein each R¹, R², and R³ is independently selected from the group consisting of hydrogen or a branched- or straight chain alkyl group having 1 to 6 carbon atoms,
X is an O or NR⁶ group, wherein R⁶ is hydrogen or a branched- or straight-chain alkyl group having 1 to 6 carbon atoms;
m and n are independently selected from an integer from 1 to 10, and Y is a pharmaceutically-acceptable anion; and
wherein X is directly bonded to the one or more protease inhibitors or X is indirectly bonded to the one or more protease inhibitors through a linker comprising up to 25 carbon atoms.

54. The pharmaceutical composition of claim 53, wherein m and n are independently selected from the integers 1 to 3.

55. The pharmaceutical composition of claim 53, wherein m and n are 1.

56. The pharmaceutical composition of claim 53, wherein X is an NH group, directly bonded to the one or more protease inhibitors.

57. The pharmaceutical composition of claim 53, wherein X is an NH group, indirectly bonded to the one or more protease inhibitors through the linker.

58. The pharmaceutical composition of claim 53, wherein the carrier molecule or a pharmaceutically acceptable salt thereof has the structure 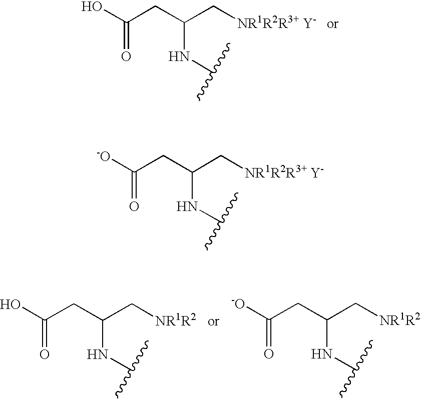
wherein R¹, R², and/or R³ are independently selected from the group consisting of hydrogen or C₁-C₃ alkyl groups, Y⁻ is a pharmaceutically acceptable anion, and the NH group is directly or indirectly bonded to the protease inhibitor.

59. The pharmaceutical composition of claims 53 and 58, wherein R¹, R², and R³ are methyl.

60. The pharmaceutical composition of claim 53 wherein the carrier molecule, or a pharmaceutically acceptable salt thereof, has the structure 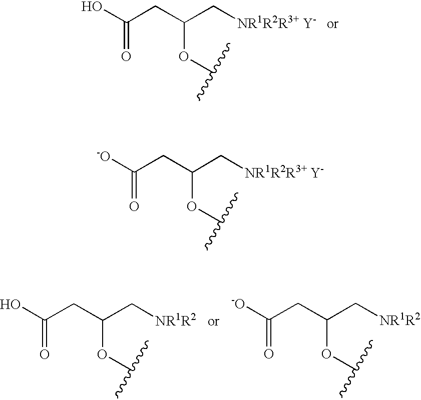
wherein R¹, R², and/or R³ are independently selected from the group consisting of hydrogen or methyl groups, Y⁻ is a pharmaceutically acceptable anion, and the indicted oxygen atom is directly or indirectly bonded to the protease inhibitor.

61. The pharmaceutical composition of claim 53, 58, and 60 wherein the protease inhibitor comprises an acetal prodrug of a peptide-aldehyde protease inhibitor.

62. The pharmaceutical composition of claims 53, wherein the protease inhibitor has the structure 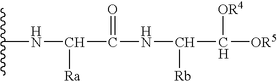
wherein R⁴ and R⁵ are a branched- or straight chain alkyl group having one to three carbon atoms, or wherein R⁴ and R⁵ together form an alkylene residue that forms a five or six membered ring, and wherein Rₐ is hydrogen or a C₁-C₄ alkyl, and R_{b} is hydrogen, a C₁-C₄ straight or branched alkyl, or has one of the structures 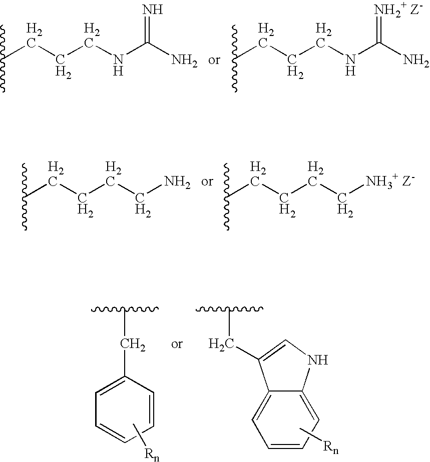
wherein Rₙ is hydrogen, hydroxyl, methyl, ethyl, methoxy, —NH₂, —NHCH₃, —N(CH₃)₂, —SH, or —SCH₃, and wherein Z- is a pharmaceutically acceptable anion.

63. The pharmaceutical composition of claim 62, wherein Rₐ is an isopropyl, isobutyl, or 2-butyl group.

64. The pharmaceutical composition of claim 62, wherein R_{b} is an isopropyl, isobutyl, or 2-butyl group.

65. The pharmaceutical composition of claim 62, wherein R_{b} has the structure

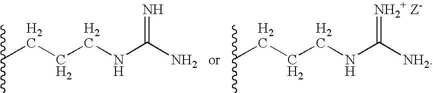

66. The pharmaceutical composition of claim 62, wherein R_{b} has the structure

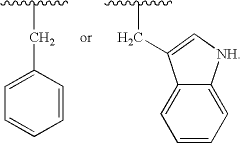

67. The pharmaceutical composition of claim 62, wherein R⁴ and R⁵ are ethyl.

68. The pharmaceutical composition of claims 53 and 58, wherein the protease inhibitor has the structure 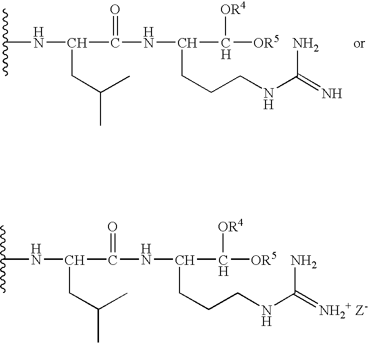
wherein R⁴ and R⁵ are a branched- or straight chain alkyl group having one to three carbon atoms, or wherein R⁴ and R⁵ together form an alkylene residue that forms a five or six membered ring, and wherein Z- is a pharmaceutically acceptable anion.

69. The pharmaceutical composition claim 69, wherein R⁴ and R⁵ are ethyl.

70. The pharmaceutical composition of claim 53, 58, and 60 wherein the linker comprises a polyalkylene group, a polyether group, a polyamide group, a polyimino group, a polyester, an aryl group, or a polythioether group.

71. The pharmaceutical composition of claims 53 and 58, wherein the linker has the formula —C¹(O)(CH₂)ₒ(O)C²—, wherein o is an integer of from 1 to 10, wherein C¹ is covalently bonded to the carrier molecule and C² is covalently bonded to the protease inhibitor.

72. The pharmaceutical composition in claim 71, wherein o is from 1 to 5.

73. The pharmaceutical composition in claim 71, wherein o is 2.

74. The pharmaceutical composition of claim 53, wherein the prodrug has the formula III 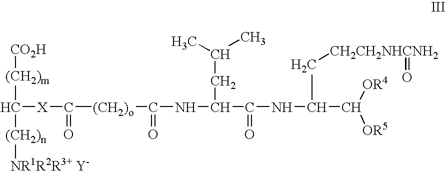
wherein each R¹-R³ is, independently, hydrogen or a branched- or straight chain alkyl group,
R⁴ and R⁵ are, independently, hydrogen or a branched- or straight chain alkyl group, or
R⁴ and R⁵ can be part of a ring,
X is O or NR⁶, wherein R⁶ comprises hydrogen or a branched- or straight-chain alkyl group;
Y and Z are a pharmaceutically-acceptable anion; and
m, n, and o can be an integer from 1 to 10,
or a pharmaceutically-acceptable salt or ester thereof.

75. The pharmaceutical composition of claim 74, wherein m and n are 1, and o is 1, 2, or 3.

76. The pharmaceutical composition of claim 74, wherein m and n are 1, R¹-R³ are methyl, and X is NH.

77. The pharmaceutical composition of claim 74, wherein the compound has the formula III, wherein m and n are 1, R¹-R³ are methyl, R⁴ and R⁵ are ethyl, X is NH, and o is 2.

78. The pharmaceutical composition of claim 55, wherein the one or more pharmaceutically acceptable carriers comprise water.

79. The pharmaceutical composition of claim 55, wherein the one or more pharmaceutically acceptable carriers comprise water.

80. A method for treating or preventing Duchenne muscular dystrophy in a mammal, comprising administering to the mammal the pharmaceutical composition of claims 53, in an amount effective to treat or prevent Duchenne muscular dystrophy in the mammal.

81. A method for treating or preventing Duchenne muscular dystrophy in a mammal, comprising administering to the mammal the pharmaceutical composition of claim 58, in an amount effective to treat or prevent Duchenne muscular dystrophy in the mammal.

82. A method for treating or preventing Duchenne muscular dystrophy in a mammal, comprising administering to the mammal the pharmaceutical composition of claim 63, in an amount effective to treat or prevent Duchenne muscular dystrophy in the mammal.

83. A method for treating or preventing Duchenne muscular dystrophy in a mammal, comprising administering to the mammal the pharmaceutical composition of claim 75, in an amount effective to treat or prevent Duchenne muscular dystrophy in the mammal.

84. A method for treating or preventing Duchenne muscular dystrophy in a mammal, comprising administering to the mammal the pharmaceutical composition of claim 78, in an amount effective to treat or prevent Duchenne muscular dystrophy in the mammal.

85. The method of claim 84, wherein after administration of the acetal prodrug of the compound to the subject, the acetal prodrug is taken up into muscle cells by an active or passive transport system.

86. The method of claims 84, wherein after administration of the acetal prodrug of the compound to the subject, the acetal prodrug is converted in-vivo to the aldehyde form of the compound.

87. The method of claims 84, wherein upon administration of the acetal prodrug form of the compound intravenously to C57BL/10SNJ mice at a concentration of about 1 mg/kg, and after approximately 4 hours, the concentration of the aldehyde form of the compound in the muscles of the mouse is at least 15% of the concentration of acetal prodrug form of the compound, as measured by HPLC/Mass Spec analysis of filtered aqueous solutions of muscle tissue homogenates.

88. The method of claims 84, wherein the acetal prodrug form of the compound, when administered to intravenously to C57BL/10SNJ mice at a concentration of about 1 mg/kg, and after approximately 4 hours, the concentration of the compound in the muscles is at least twice as high as the concentration of the compound in liver, as measured by HPLC/Mass Spec analysis of filtered aqueous solutions of tissue homogenates.

89. The method of claim 88, wherein the aldehyde form of the compound, when present at concentration of about 1.0 millimolar, inhibits the proteolytic activity of m-calpain by at least 20%.

90. A method for treating or preventing Duchenne muscular dystrophy in a mammal, comprising administering to the mammal one or more aldehyde compounds, or pharmaceutically acceptable salts thereof, in an amount effective to treat or prevent Duchenne muscular dystrophy in the mammal, wherein the aldehyde compound has the structure: 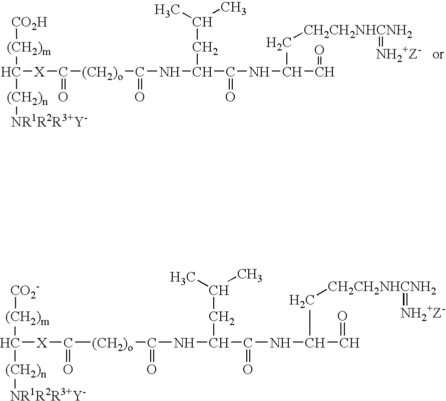
wherein each R¹-R³ is, independently, hydrogen or a branched- or straight chain alkyl group having 1 to 6 carbon atoms,
X is O or NR⁶, wherein R⁶ comprises hydrogen or a branched- or straight-chain alkyl group having 1 to 6 carbon atoms;
Y and Z are pharmaceutically-acceptable anions; and
m, n, and o can be an integer from 1 to 10,
or the pharmaceutically-acceptable salt or ester thereof.

91. The method of claim 90, wherein m and n are 1, and 0 is 1, 2, or 3.

92. The method of claim 90, wherein R¹-R³ are methyl.

93. The method of claim 90, wherein m and n are 1, R¹-R³ are methyl, and X is NH.

94. The method of claim 90 wherein m and n are 1, R¹-R³ are methyl, X is NH, and o is 2.

95. The method of claim 91, wherein the aldehyde compound is administered in an amount of at least about 0.1 mg/kg/day.

96. The method of claim 91, wherein the mammal is a human male diagnosed with Duchenne muscular dystrophy.

97. A compound having the structure 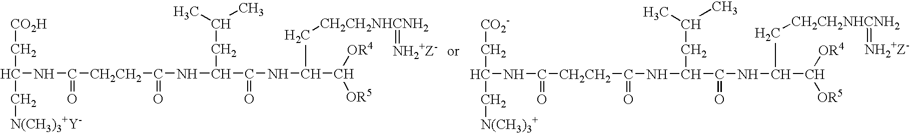
or a pharmaceutically acceptable salt thereof, wherein Y and Z are pharmaceutically-acceptable anions.

98. A pharmaceutical composition comprising one or more pharmaceutically acceptable carriers and the compound of claim 97.

99. A method for treating or preventing Duchenne muscular dystrophy in a human diagnosed with Duchenne muscular dystrophy, comprising administering to the subject an amount of the compound of claim 97 effective to treat or prevent Duchenne muscular dystrophy.

100. The use of the compound of claim 97 for the manufacture of a medicament for treating Duchenne muscular dystrophy.

101. A compound having the structure 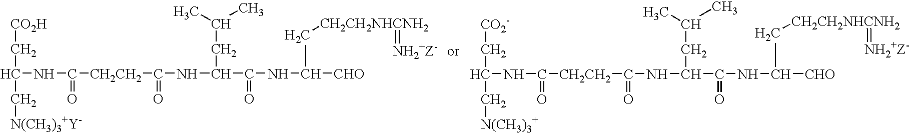
or a pharmaceutically acceptable salt thereof, wherein Y and Z are pharmaceutically-acceptable anions.

102. A pharmaceutical composition comprising one or more pharmaceutically acceptable carriers and the compound of claim 101.

103. The use of the compound of claim 101 for the manufacture of a medicament for treatment or prevention of Duchenne muscular dystrophy.

104. A method for treating or preventing Duchenne muscular dystrophy in a human diagnosed with Duchenne muscular dystrophy, comprising administering to the subject an amount of the compound of claim 101 effective to treat or prevent Duchenne muscular dystrophy. 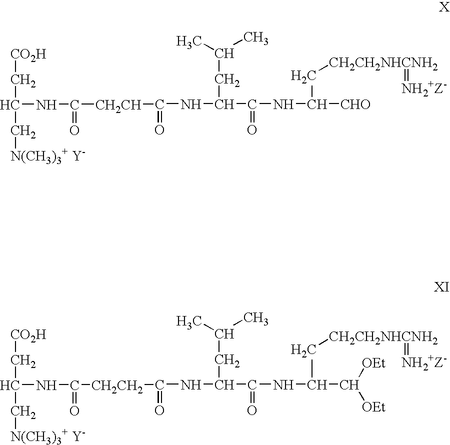
wherein Y and Z comprises a pharmaceutically-acceptable anion.
